(19) United States
(12) Patent Application Publication
(10) Pub. No.: US 2023/0166255 A1
(43) Pub. Date: Jun. 1, 2023

(54) Methods And Devices For Detection Of Anticoagulants In Plasma And Whole Blood

(71) Applicants: Massachusetts Institute of Technology, Cambridge, MA (US); The General Hospital Corporation, Boston, MA (US)

(72) Inventors: Galit H. Frydman, Boston, MA (US); Mehmet Toner, Charlestown, MA (US); Ronald G. Tompkins, Boston, MA (US); Pavan Bendapudi, Boston, MA (US)

(21) Appl. No.: 18/159,281

(22) Filed: Jan. 25, 2023

Related U.S. Application Data
(62) Division of application No. 16/046,816, filed on Jul. 26, 2018, now abandoned.
(60) Provisional application No. 62/538,618, filed on Jul. 28, 2017.
(60) Provisional application No. 62/699,665, filed on Jul. 17, 2018.

(51) Int. Cl. B01L 3/00; G01N 33/86; G01N 33/49; A61K 38/48

(52) U.S. Cl. CPC B01L 3/502746; A61K 38/4846; G01N 33/86; G01N 33/4905; B01L 2300/165; B01L 2400/088; B01L 2400/0406

(57) ABSTRACT

Methods and devices for evaluating coagulation are described, including methods and devices for detecting an anticoagulant agent or a coagulation abnormality. In various embodiments, the methods and devices of the invention measure coagulation of a sample in response to a gradient of one or more coagulation factors. These responses can be evaluated to accurately profile coagulation impairments of the sample, including the presence of anticoagulant medication. In various embodiments, the invention provides point-of-care or bedside testing with a convenient, microfluidic device that can be used by minimally trained personnel.

### RELATED APPLICATIONS

This application is a divisional of U.S. Application No. 16/046,816, filed Jul. 28, 2018, which claims the benefit of U.S. Provisional Application No. 62/538,618, filed on Jul. 28, 2017, and U.S. Provisional Application No. 62/699,665, filed on Jul. 17, 2018, the entire contents of which are hereby incorporated by reference.

### GOVERNMENT SUPPORT

This invention was made with Government support under Grant Nos. P41 EB002503, P30 ES002109, and P50 GM021700 awarded by the National Institutes of Health. The Government has certain rights in the invention.

### BACKGROUND

The coagulation system is a delicate balance between hemorrhage and thrombosis. There are many disease states, including cancer, auto-immune disease, infection, trauma, surgery, heart disease, and drugs, that can cause a disruption of this balance and result in a patient having severe, even life-threatening, bleeding or clotting events. Anticoagulant medications are commonly prescribed for thrombotic disorders. Conventional anticoagulant medications, such as Heparin, will indirectly inhibit multiple factors of the clotting cascade. The more recent introduction of direct oral anticoagulants (DOACs) allows for targeted inhibition of the coagulation pathway.

The biggest risk of anticoagulation therapy is the increased risk of bleeding, and thus, traditionally, patients taking anticoagulant medications are carefully monitored to ensure that they are receiving an appropriate dose. Current clinical tests available to evaluate a patient’s bleeding and clotting are either rudimentary and provide very vague information, such as prothrombin time (PT) and activated thromboplastin time (aPTT), or are more detailed but require expensive machines, lengthy training, and careful handling. Included in the latter category are thromboelastography (TEG), thromboelastometry (TEM), rotational thromboelastometry (ROTEM), platelet aggregometry and flow cytometry. Currently, specific tests for the DOACs are not available. Most of the DOAC assays that have been proposed are pharmacokinetic assays that measure the absolute concentration of the drug itself and, therefore, provide limited functional information to support clinical decision-making.

Coagulation tests are needed that can detect, characterize, and/or quantify impairments in coagulation, including detection of DOACs in patient samples, to better manage patients at high risk of severe bleeding or clotting, including, but not limited to, the urgent care setting.

### SUMMARY

Methods and devices for evaluating coagulation are described, including methods and devices for detecting an anticoagulation agent or a coagulation abnormality. Coagulation abnormality includes abnormality of clot formation (e.g., thrombosis) and abnormality of clot degradation (e.g., fibrinolysis). In various embodiments, the methods and devices of the invention measure coagulation of a sample in response to a gradient of one or more coagulation factors. These responses can be evaluated to accurately profile coagulation impairments of the sample, including the presence of a DOAC or traditional anticoagulant medication. In various embodiments, the invention provides point-of-care or bedside testing with a convenient, microfluidic device that can be used by minimally trained personnel.

In some aspects, the invention provides methods for assessing coagulation in a blood sample. The method comprises adding a coagulation factor to plural portions (e.g., aliquots) of the blood sample, each portion receiving the coagulation factor at a different concentration, and measuring clot formation or clot formation times in response to the different concentrations. By assessing coagulation in response to the different concentrations of one or more coagulation factors, blood clotting function can be accurately profiled, including the impact of DOACs or other drugs on coagulation. In some embodiments, the presence or absence of a genetic clotting abnormality is determined. The methods as described herein may be performed using a microfluidic device as described, where one or more of the channels can be configured to trigger formation and localization of a clot.

As used herein, unless described otherwise, a “blood sample” refers to a whole blood sample or a plasma sample. The term plasma includes both platelet-rich-plasma (PRP) and platelet-poor-plasma (PPP).

The term “coagulation factor” as used herein means any factor implicated in the coagulation cascade (intrinsic, extrinsic and common pathways), including Factors I to XIII, von Willebrand factor, prekallikrein (Fletcher factor), high-molecular-weight kininogen (HMWK) (Fitzgerald factor), fibronectin, antithrombin III, heparin cofactor II, protein C, protein S, protein Z, Protein Z-related protease inhibitor (ZPI), plasminogen, alpha 2-antiplasmin, tissue plasminogen activator (tPA), urokinase, plasminogen activator inhibitor-1 (PAI1), plasminogen activator inhibitor-2 (PAI2), Tissue Factor Pathway Inhibitor (TFPI), and cancer procoagulant. The coagulation factor(s) can be in activated form or inactivated (e.g., precursor) form. For example, for detecting the presence of a coagulation factor inhibitor in a sample, the coagulation factor should be in activated form (e.g., Factor Xa or Factor IIa). In other embodiments, for detection of a genetic clotting abnormality, the coagulation factor may be in inactivated form (e.g., Factor X or Factor II). Further, the coagulation factor(s) can be from a human, an animal (such as bovine, porcine or other), or can be a synthesized or recombinant protein.

In some embodiments, the invention provides a method of detecting an anticoagulation agent. Anticoagulation agents are substances that prevent or reduce coagulation of blood, prolonging clotting time. Anticoagulation agents include, but are not limited to, Factor-specific inhibitors (such as FXa inhibitors, FIIa inhibitors, FXIa inhibitors, FXIIa inhibitors), heparins, and vitamin K antagonists (e.g., warfarin). In some embodiment, they include Direct Oral Anticoagulants (DOACs), also known as Novel Oral Anticoagulants (NOACs), such as XARELTO (Rivaroxaban) by Janssen Pharmaceuticals, Inc., ELIQUIS (Apixaban) by Bristol-Myers Squibb and Pfizer Inc., SAVAYSA (Edoxaban) by Daiichi Sankyo, Inc., PRADAXA (Dabigatran) by Boehringer Ingelheim, and BEVYXXA (Betrixaban) by Portola Pharmaceuticals, Inc.

By measuring clot formation (e.g., clot formation times) in response to increasing concentrations of exogenously added coagulation factors, the presence and/or point of inhibition by a therapeutic agent can be determined. For example, a sample that is positive for a coagulation inhibitor will show a concentration-dependent decrease in clotting time as the coagulation factor that is targeted by the inhibitor is added to the sample. Meanwhile, when a coagulation factor upstream from the point of inhibition is added (in increasing amounts), the clotting time will remain prolonged, as compared to the clotting time upon the addition of a coagulation factor downstream of the point of inhibition. See FIGS. 9-13.

In some embodiments, results for a patient sample can be compared to reference standards, including standards for normal and/or abnormal clotting, or reference standards corresponding to anticoagulant therapy with particular agents. In some embodiments, reference standards are personalized for the patient.

In various embodiments, clotting curves can be constructed to characterize the response of clot formation to the addition of various coagulation factors in increasing concentrations or amounts. These clotting curves allow for the identity and amount of coagulation inhibitors to be determined, to thereby guide patient care. In some embodiments, the appropriate coagulation inhibitor reversal agent is then administered to the patient to reverse the therapeutic intervention as needed.

In some aspects, the invention provides a microfluidic device for evaluating coagulation in a sample. The device includes a series of channels in a substrate, each channel having an area with a geometry to trigger and/or localize formation of a clot, to allow for evaluation of clot formation in response to one or more reagents, such as the amount or concentration of an exogenously added coagulation factor. The channels in the series each have the same geometry, so as to trigger identical clot formation properties (when exposed to the same sample and reagents). By evaluating clot formation in the presence of a gradient of one or more coagulation factors, the invention allows for sensitive and specific detection of coagulation abnormalities or impairments, including the presence or activity of a DOAC in the sample.

In one embodiment, the microfluidic device for detecting coagulation includes plural channels formed in a substrate, each channel including a clot forming area having a geometry configured to trigger and/or localize formation of a clot. The clot forming areas of the plural channels may be arranged in a central region of the substrate in some embodiments, such that the clotting properties can be simultaneously imaged or analyzed across the channels. See FIGS. 1A-1B, 2B. The device may further include plural sample input ports to receive a sample (e.g., whole blood or plasma), each sample input port connected to a first end of one of the plural channels. See FIGS. 1A-1D. In other embodiments, the device has a single sample input port in fluid communication with the plural channels, or a series of channels. See FIG. 5A. In some embodiments, each channel has an independent output port, each output port connected to a second end of one of the plural channels. In embodiments employing independent sample input ports, the input and output ports can be arranged in an alternating pattern at a periphery of the substrate. See FIGS. 1A-1B, 2A. In some embodiments, the input and output ports are arranged in a pattern other than an alternating pattern.

The term “central region” as used herein means a region that is located in the center of a substrate relative to a periphery of the substrate and can include a region that is positioned off-center. For example, depending upon the configuration, the central region might be off-center and the areas in the microfluidic channels in which clots begin can be controlled by the flow patterns in the channels.

In some embodiments, the clot forming areas of the plural channels are arranged in a region of the substrate which is not central, such as, but not limited to, the periphery. See FIGS. 5A-5B.

Each channel may further comprise one or more additional input ports to receive reagents, such as coagulation factor(s) and/or calcium. In some embodiments, there is more than one input port (e.g., for introducing sample and one or more reagents) per output port. For example, in one embodiment, there can be one input port for the sample and 1 to 2 input ports for the reagents (e.g., coagulation factor and, optionally, calcium). See FIG. 1B. In some embodiments, there is one common input port for the sample, and each channel further comprises further input ports (e.g., 1 or 2) for reagents.

In the microfluidic device, each clot forming area can be configured to create an area of stasis or disruption in fluid flow to trigger and/or localize formation of a clot. In some embodiments, each clot forming area can be configured to create an area of flow disturbance to trigger and/or localize clot formation. Exemplary geometries for triggering formation of and localizing a clot are illustrated in FIGS. 2B, 3A, 5A and 5B.

Channels of the microfluidic device can be coated with, contain or otherwise include a coagulation factor at a different amount or concentration. For example, a first group or series of the plural channels can be coated with, contain or otherwise include a first coagulation factor, and a second group or series of the plural channels can be coated with, contain or otherwise include a second coagulation factor. Further, in some embodiments, one of the plural channels is a negative control channel, e.g., may not be coated with and may not include a coagulation factor. In other embodiments, the device does not comprise such a negative control channel.

In the case where one or more channels include the coagulation factor(s), the coagulation factor(s) may be in suspension or solution, or lyophilized and not surface-bound. The coagulation factor(s) can be pre-included in the channel(s) (e.g., at the time of manufacturing the device), can be added prior to placing the sample into the device, or can be entered into the device through an input port (or multiple input ports) simultaneously with the sample or after the sample.

In embodiments of the microfluidic device that include first and second groups of channels (whether or not such embodiments may also include a negative control channel in addition to the first and second groups of channels), each channel in the first group of the plural channels can be coated with, contain or otherwise include a first coagulation factor at a different amount or concentration, and each channel in the second group of the plural channels can be coated with, contain or otherwise include a second coagulation factor at a different amount or concentration. In some embodiments, the microfluidic device may contain more than two groups or series of plural channels, such as three, four, five or more groups, wherein each group or series of plural channels is coated with, contains or otherwise includes a different coagulation factor at an increasing amount across the group or series (e.g., a microfluidic device containing four groups of channels, each group of the plural channels can be coated with, contain or otherwise include a different coagulation factor selected from Factors IIa, Xa, XI, XIa, XII, and XIIa). By measuring clot formation or clotting time as a function of coagulation factor gradients, the sample’s clotting properties can be profiled at several specific points of the coagulation pathway(s) (illustrated in FIG. 8), providing a clinician with detailed and specific information concerning the patient’s clotting physiology and/or the status of any therapeutic intervention.

The second coagulation factor can be upstream in the coagulation cascade from the first coagulation factor. For example, the first coagulation factor can be, e.g., prothrombin (Factor II), thrombin (Factor IIa), or both. The second coagulation factor can be, e.g., Factor X, Factor Xa, or both.

The microfluidic device can further include a detection device configured to measure clot formation times in each of the channels to assess coagulation based on the clot formation times measured. For example, the detection device can be configured to image the clot forming areas simultaneously to measure clot formation times. In some embodiments, the degree of clot formation in each of the channels is quantified at a fixed time or times. For example, the detection device in connection with the methods and devices described herein can include a microscope and an image sensor. Imaging the clot forming areas can include bright-field imaging. For the devices and assays described herein, clotting times can also be measured with other methodologies such as detection based on light absorbance, fluorescence measurements, ultrasound, etc., and the detection device can be configured to employ one or more of these other methodologies. Ways to detect clotting also include, but are not limited to, detection based on electrical impedance, the addition of beads and quantifying bead flow rate/number, measurement of flow velocity and/or pressure before and/or after the site of clot formation, thromboelastography, fluorescence detection (such as with fluorescent fibrinogen), turbidity, magnetic, flow dynamics (pressure or flow velocity), infrared light detection, infrared spectroscopy, detection using acoustic and/or photonic sensors, flow cytometry, and visual clotting detection.

In some embodiments, the method described herein does not employ a microfluidic device, but uses wells or containers suitable for inducing and measuring formation of a clot.

In addition to clot formation times, other characteristics of clot formation can be considered. It is contemplated that a qualitative measure of clot formation, in addition to clot formation times, can be useful, e.g., to determine the most sensitive detection mode for coagulation. For example, properties of the clot such as size, strength, density and composition can be assessed in addition to time to form a clot. Such properties may be assessed using the same or a different detection modality than is used to detect clot formation times.

In some embodiments, clot lysis can be assessed in addition to clot formation. For example, if a patient is on a fibrinolytic or thrombolytic agent, one can evaluate the clot when it is being formed as well as its breakdown over time. In one embodiment, the same methods described herein and known in the art to detect clot formation can be used to assess clot lysis over time.

As described herein regarding the use of thromboelastography (TEG), one can evaluate both clot formation and fibrinolysis. This would be useful for detecting clotting abnormalities in patients that are hypocoagulable due to problems with fibrinolysis or iatrogenic administration of fibrinolytic and thrombolytic drugs. See, for example, C. Mauffrey, et al., “Strategies for the management of haemorrhage following pelvic fractures and associated trauma-induced coagulopathy,” Bone Joint J. 2014; 96-B:1143-54, the relevant teachings of which are incorporated herein by reference.

In any of the devices and methods described herein, the blood sample can be a whole blood sample or a plasma sample. Using whole blood can be particularly useful for certain applications, such as those implemented at the bedside of a patient.

The disclosed devices and methods can be applied to all individuals, including mammals (e.g., humans, such as human patients, as well as non-human mammals), reptiles, birds, and fish, among others, and can be useful for research and veterinary medicine. An individual can be, for example, mature (e.g., adult) or immature (e.g., child, infant, neonate, or pre-term infant).

The disclosed devices and methods can be used not just for diagnostic purposes but also for research and discovery to explore the coagulation cascade in a research setting. For example, this can be useful for basic drug discovery, understanding disease or disorder pathophysiology, for example, in the context of hemorrhagic diseases (Dengue virus, Zika virus, Ebola virus, etc.), and also to monitor for adverse events of experimental treatments.

The disclosed devices and methods can be used to guide therapy of a patient. For example, physicians can use the results to determine subsequent treatments with both drugs and procedural interventions (both invasive and non-invasive). For example, if a patient tests positive for Factor IIa inhibition due to dabigatran administration, then the healthcare provider may choose to administer the reversal agent (idarucizumab) for this inhibitor prior to surgery or other invasive procedures. Likewise, if the patient tests positive for Factor Xa inhibition, then the healthcare provider may choose to administer the appropriate reversal agent (coagulation factor Xa (recombinant), inactivated-zhzo) for this inhibitor. The healthcare provider may choose to administer other agents that overcome the effects of these inhibitors as well, such as 4-factor prothrombin complex concentrates or activated prothrombin complex concentrates.

Other aspects and embodiments of the invention will be apparent from the following Drawings and Detailed Description.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1D are schematic illustrations of microfluidic device layouts employing multiple sample ports according to example embodiments of the invention.

FIG. 2A is a top view of the circular microfluidic clotting device according to an example embodiment.

FIG. 2B is a magnified view of the central portion of the device of FIG. 2A. FIG. 2B illustrates exemplary geometries of clot formation areas.

FIGS. 3A-3C illustrate clot detection using plasma and fluorescent-labeled fibrinogen within a microfluidic device having four channels according to an example embodiment. FIG. 3A is a top view bright-field image of a central portion of the example microfluidic device. FIG. 3B is a fluorescent image of clot formation using the device of FIG. 3A. FIG. 3C is a fluorescent image showing a magnified view of a clot forming area.

FIGS. 4A and 4B are bright-field images illustrating clot detection using whole blood in a parallel microfluidic channel device employing a FXa gradient, according to an example embodiment. FIG. 4A contains no anticoagulant. FIG. 4B contains unfractionated heparin.

FIGS. 5A and 5B are schematic illustrations of microfluidic device configurations employing a single port for sample input according to example embodiments of the invention.

FIG. 6 is a flow diagram of an assay or a method according to example embodiments of the invention.

FIG. 7A is a graph of example data illustrating detection of Rivaroxaban, using a FXa gradient.

FIG. 7B is a graph of example data illustrating detection of Apixaban, using a FXa gradient.

FIG. 7C is a graph of example data illustrating detection of Edoxaban, using a FXa gradient.

FIG. 7D is a graph of example data illustrating detection of Dabigatran, using a FIIa gradient.

FIG. 8 is a diagram illustrating a basic clotting cascade.

FIG. 9 is a diagram illustrating how to detect FXa inhibition/deficiency/abnormality of function by employing coagulation factor gradients.

FIG. 10 is a diagram illustrating how to detect FIIa inhibition/deficiency/abnormality of function by employing coagulation factor gradients.

FIG. 11 is a diagram illustrating how to detect and differentiate between FIIa and FXa inhibition in a sample by employing coagulation factor gradients.

FIG. 12 is a diagram illustrating how to detect indirect FXa inhibition/deficiency/abnormality of function by employing coagulation factor gradients.

FIG. 13 is a diagram illustrating how to detect and differentiate between FXIIa and FXIa inhibition in a sample by employing coagulation factor gradients.

FIG. 14 is a diagram illustrating how to detect and differentiate between various types of hemophilia by employing coagulation factor gradients.

FIG. 15 is a diagram illustrating how to detect problems with fibrinogen or FXIII (e.g., FXIII deficiency) by employing coagulation factor gradients.

FIGS. 16A-16C illustrate Clotting Curve Scores (CCS) for FXa and FIIa inhibitors at various concentrations.

FIG. 17 shows Table 1 of patient descriptive statistics (Example 17).

FIGS. 18A-18C illustrate measurements of sensitivity and specificity of prothrombin time (PT) (FIG. 18A) and international normalized ratio (INR) (FIG. 18B) for FXa inhibitor (FXa-I) anticoagulation.

FIGS. 19A-19G illustrate example clotting time data and comparative clotting curves.

FIGS. 20A-20E illustrate Clotting Curve Score (CCS) analysis and evaluation of CCS utilization for the detection of FXa-I in patient samples.

FIGS. 21A and 21B illustrate example functional drug concentration calculation.

FIG. 22 illustrates a current decision-making paradigm for a patient that is bleeding or at high risk.

FIG. 23 illustrates an improved decision-making paradigm using embodiment(s) of the present invention for a patient that is bleeding or at high risk.

FIGS. 24A and 24B illustrate detection of decrease in FXa inhibition by a FXa-I after the addition of activated prothrombin complex concentrate (aPCC).

### DETAILED DESCRIPTION

The invention generally relates to methods and devices for the detection of coagulation, including detection of coagulation abnormalities and detection of anticoagulants and platelet inhibitors in plasma and/or whole blood.

Acquired coagulopathies are a major component of morbidity and mortality in a number of medical settings. Individuals may have increased risk of internal bleeding secondary to drugs (e.g., clopidogrel, heparin, warfarin or other vitamin K antagonists, dabigatran or other Direct Oral Anticoagulants, etc.), trauma, surgery, sepsis, cancer, organ dysfunction (e.g., liver), or congenital abnormality (e.g., hemophilia). On the other end of the spectrum, increased propensity for clotting can be due to autoimmune disease, cancer, atherosclerosis, early trauma and sepsis, organ dysfunction (e.g., kidney), immobility, inflammation, foreign body (e.g., stent or prosthesis), or congenital abnormality (e.g., Factor V Leidin thrombophilia). With recent innovations in drug development (e.g., anticoagulants, including Direct Oral Anticoagulants, or DOACs), innovation is now needed for hemostasis/coagulation analyzers, to fully realize benefits for patients, including in the urgent care setting. Specifically, current clinical tests available to evaluate a patient’s bleeding and clotting are either rudimentary and provide very vague information, such as prothrombin time (PT) and activated thromboplastin time (aPTT), or are more detailed but require expensive machines, lengthy training, and careful handling, such as thromboelastography (TEG), thromboelastometry (TEM), rotational thromboelastometry (ROTEM), platelet aggregometry and flow cytometry. Currently, specific tests for DOACs are not available. Most of the DOAC assays that have been proposed are pharmacokinetic assays that measure the absolute concentration of the drug itself and, therefore, provide limited functional information for clinical decision-making.

With the increased use of DOACs, studies and reviews are finding that, although these new drugs pose less risk for acute, life-threatening bleeding events, they are potentially linked to higher rates of gastrointestinal (GI) bleeding. Additionally, these new drugs are found to have different pharmacokinetic properties in patients with decreased liver and/or kidney function or in patients that are on multiple drugs at the same time, as is common in the geriatric population. In these cases, providing functional clinical information to the doctor to help personalize the anticoagulant combination and dosage would be of great benefit to the patient and possibly decrease subsequent, related adverse events. Embodiments of the invention can be used in clotting panels that evaluate the coagulation, fibrinolysis, and platelet function within an individual. The microfluidic technology and advanced assays described herein in some embodiments provide for custom clotting panels, whereby clinicians can determine a patient’s coagulation function bedside. These embodiments provide for vast improvements in patient care, including in the urgent care setting.

In addition to these assays being rapid and easy-to-interpret, they can also be customizable, allowing for the selection of clinically-relevant coagulation and platelet function testing for each customer and/or end-user segment. Because embodiments of the assay can be applied in a bedside platform, it can also be utilized for trend-monitoring in patients on various treatments (including at the hospital, at anticoagulation clinics and at home). In an aspect of the invention, a gradient of the factor(s) is added to the sample after it is subdivided into and/or distributed among the multiple groups of plural channels, wells, or containers, which method permits evaluation of coagulation function/inhibition and identification and differentiation between various coagulation abnormalities within a sample. This means that embodiments of the invention (e.g., clotting panels, assays, etc.) are potentially useful for assessing coagulation in patients that have poor medical compliance, where dosage/time taken is unknown, or in patients that are unconscious, where the doctor, surgeon or other healthcare provider needs to know whether the patient has any of these drugs in their system. Further, embodiments can help in monitoring anticoagulation and guiding the administration of reversal reagents that are now becoming available.

Examples of potential users for product or services based on embodiments of the invention can range from healthcare workers, e.g., clinicians and veterinarians, to researchers in pharmaceutical research and development.

The invention can be applied to patient care in various settings. In some embodiments, the patient is scheduled for surgery or is in need of an invasive procedure, and the methods and devices of the invention can be used for clinical decision-making, including preparing the patient for the procedure to minimize bleeding risks. In some embodiments, the patient is administered a drug that impacts coagulation, and the methods and devices of the invention can be used for early evaluation of drug action and for selection of the appropriate therapy and dose. In some embodiments, the patient receives a drug or blood product, and methods and devices of the invention can be used to guide administration and dose. In some embodiments, the patient has or is suspected of having, or is at risk of acquiring, a hemorrhagic virus. In some embodiments, the patient is a neonate, where only small volumes of blood are available for evaluating coagulation (including for administering anticoagulant therapy or for detecting a congenital coagulation abnormality). In some embodiments, the patient is a pregnant mother, and the methods and devices allow for detecting a congenital coagulation abnormality, or for early diagnosis of a condition that results in a coagulation abnormality such as pre-eclampsia and eclampsia.

In some embodiments, the patient or subject is a veterinary or animal patient (e.g., such as a dog, cat, or horse). In some embodiments, the patient is a non-human mammal. The cost-restrictions and limited blood volume of veterinary patients and laboratory animal research result in a large need for coagulation diagnostics that are easy-to-use, require only microliters of blood, and have lower overhead costs.

Due to the immense interest in novel coagulation testing platforms, the blood-testing platform (e.g., assay, microfluidic device, and/or combination thereof) described herein offers tremendous potential for research and product development.

In some embodiments, the patient is receiving an anticoagulant therapy, such as a heparin or vitamin K antagonist (e.g., warfarin). In some embodiment, the patient is undergoing therapy with a Direct Oral Anticoagulant (DOAC), such as XARELTO (Rivaroxaban), ELIQUIS (Apixaban), SAVAYSA (Edoxaban), PRADAXA (Dabigatran), or BEVYXXA (Betrixaban). In some embodiments, the patient is undergoing therapy with an antibody against TFPI. Anticoagulant drugs are used commonly in many medical settings, including emergency and critical care, surgery, cardiology, and cancer. Several new anticoagulants have been introduced, but there are no current tests that can reliably determine if a patient is on the right dose. Too much anticoagulation can cause life-threatening bleeding and too little can lead to an increased risk of stroke and heart attacks. Embodiments of the invention can be used as or incorporated into a bedside test that can accurately monitor these new anticoagulants and improve the safety for these patients. This test can be performed with minimal training and in an easy to interpret format. In an embodiment, these assays can be performed in the lab in a device requiring less than about 1 mL, or less than about 500 µL, or less than about 100 µL, or less than about 50 µL (one drop) of fresh or citrated, whole blood, with the results being read within 10 minutes.

The Direct Oral Anticoagulant (DOAC) market currently consists of drugs that selectively target specific factors within the coagulation pathways, e.g., Factor IIa or Factor Xa. While these drugs are very potent, because of the dearth of reliable or easy-to-use diagnostic and monitoring tests, there is an increased risk associated with the use and administration of these drugs, especially in the critical care setting. One of the primary risks of DOAC use is gastrointestinal bleeding. These adverse events not only lead to morbidity and mortality but also result in increased medical costs and longer hospitalization times.

In some embodiments, the method involves detecting a coagulation abnormality in a blood sample, and pinpointing where it occurs within the coagulation cascade, by comparing the clot formation times determined to coagulation factor-specific clot formation reference ranges, e.g., from individual(s) who do not suffer from a coagulation cascade abnormality. In some embodiments, the reference ranges can be established using the detection method on a normal subject or subjects, e.g., individuals who do not suffer from a coagulation abnormality. In some embodiments, the reference range can be established based on the same individual from whom the test blood sample(s) is obtained. For example, the reference range can be established prior to commencement of a medical treatment of an individual, and the test sample can be obtained from the same individual after the commencement of a treatment. The sample can also be obtained from a relative (e.g., parent, sibling or offspring) of the individual from whom the test sample is obtained. The reference ranges may be tailored to or dependent on a particular assay configuration, including microfluidic device configuration. In some embodiments, each subject’s clotting can be compared to a “normal” control at the testing time or to previously-determined “normal” reference ranges for the specific coagulation factor or combination of factors. In some embodiments, the assay approach requires the establishment and/or verification of reference ranges.

In some embodiments, reference ranges are from controls or standards of a specific coagulation cascade abnormality, such as from individuals who do not suffer from a coagulation cascade abnormality. In some embodiments, the reference ranges are from spiked or depleted samples/controls, which can be commercially available.

It should be understood that one can also compare clot formation times to reference ranges from someone who does suffer from a coagulation abnormality. For example, it is common with reference intervals to have a “normal” interval range for people who do not suffer from an abnormality and an “abnormal” interval range for people confirmed to have that abnormality. Sometimes, there is a gray zone in-between the normal and abnormal zones, that is indicative that further in-depth testing needs to be done on that patient sample for a definitive diagnosis.

In some embodiments, the invention does not require comparison to a reference range or standard and, instead, provides internal controls by evaluating coagulation factors upstream and downstream of a suspected point of inhibition in the coagulation pathway(s).

A description of example embodiments follows.

Embodiments described herein include rapid assays (e.g., <30 minutes, <20 minutes, <15 minutes, or <10 minutes in some embodiments) for the detection of anticoagulants and platelet inhibitors in whole blood or plasma and the assessment of patient coagulation status. The availability of these customizable coagulation panels fills an unmet need within various coagulation testing environments by providing rapid, bedside diagnostics and drug monitoring capabilities.

In an embodiment, the method includes an assay wherein a specific coagulation factor suspected of being inhibited is added into a blood sample (e.g., a whole blood or plasma sample), in various concentrations or amounts. For example, the coagulation factor can be added to divided portions of the sample in amounts that vary by a factor of 2 to a factor of 100. In some embodiments, coagulation factor is added to divided portions of the sample at concentrations increasing by a factor of 5 to a factor of 20 (e.g., about a factor of 10) across the divided portions. In some embodiments, the concentration of the coagulation factor added to the divided portions of the sample can be in the range of 0.1 ng/mL to 10 µg/mL. The addition of the coagulation factor at specific concentrations or amounts (e.g., a gradient or multiple samples with different concentrations) enables determination of:
a) The presence of a specific abnormality at this specific point of the coagulation cascade (e.g., drug-induced via an anticoagulant, auto-immune, or genetic, such as in hemophilia); and
b) The inhibition of coagulation function at this specific point of the coagulation cascade.

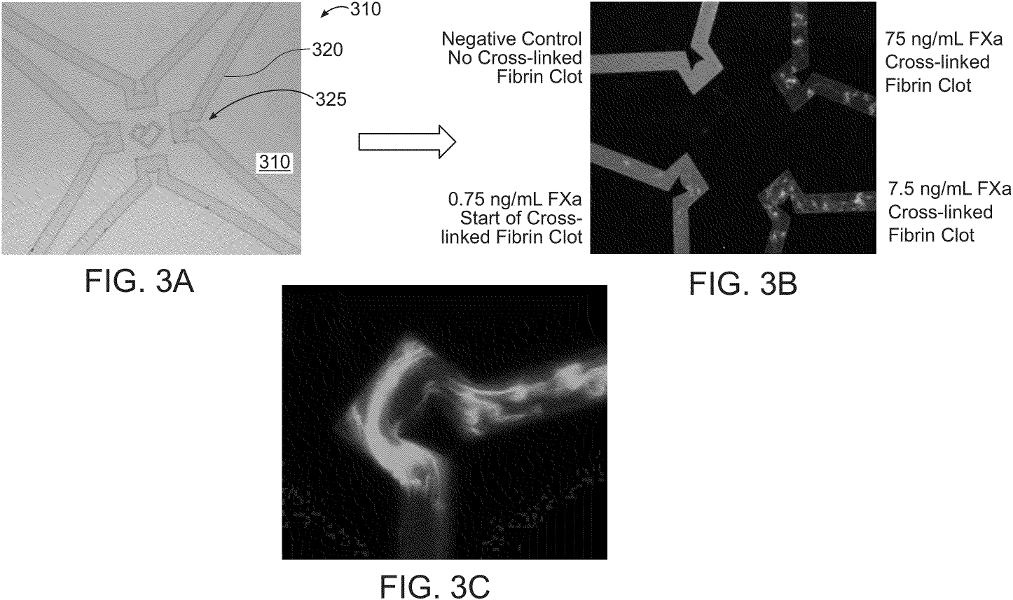

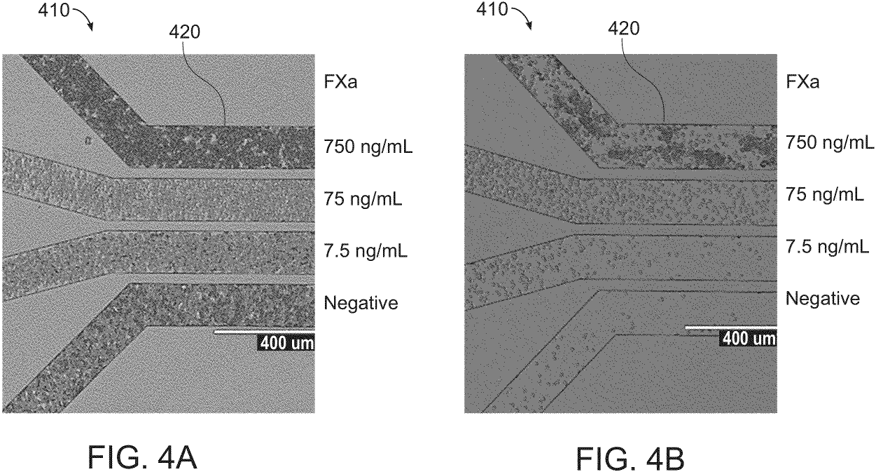

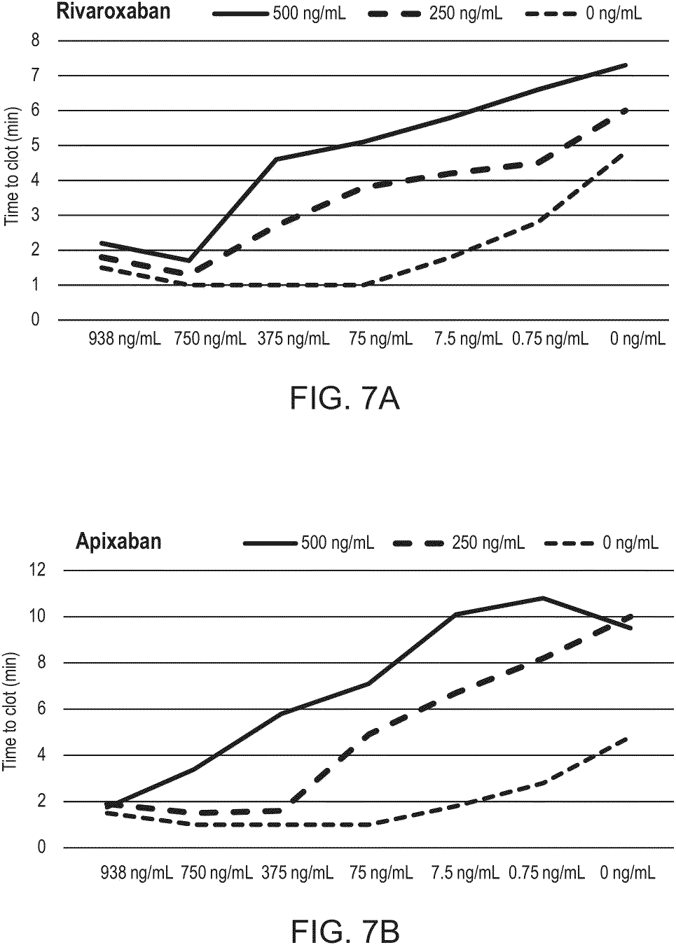

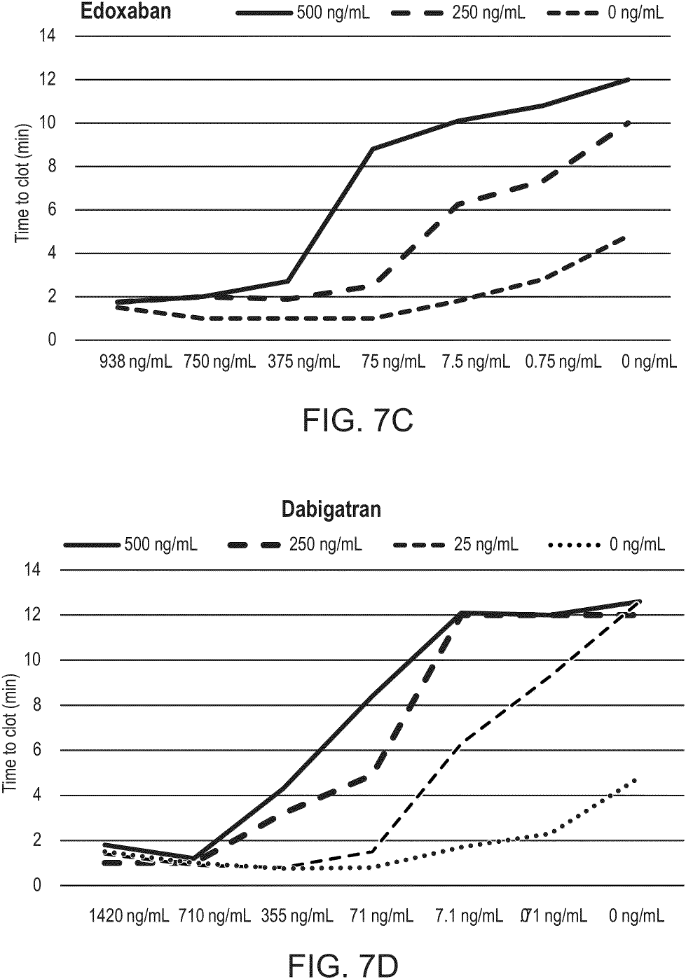

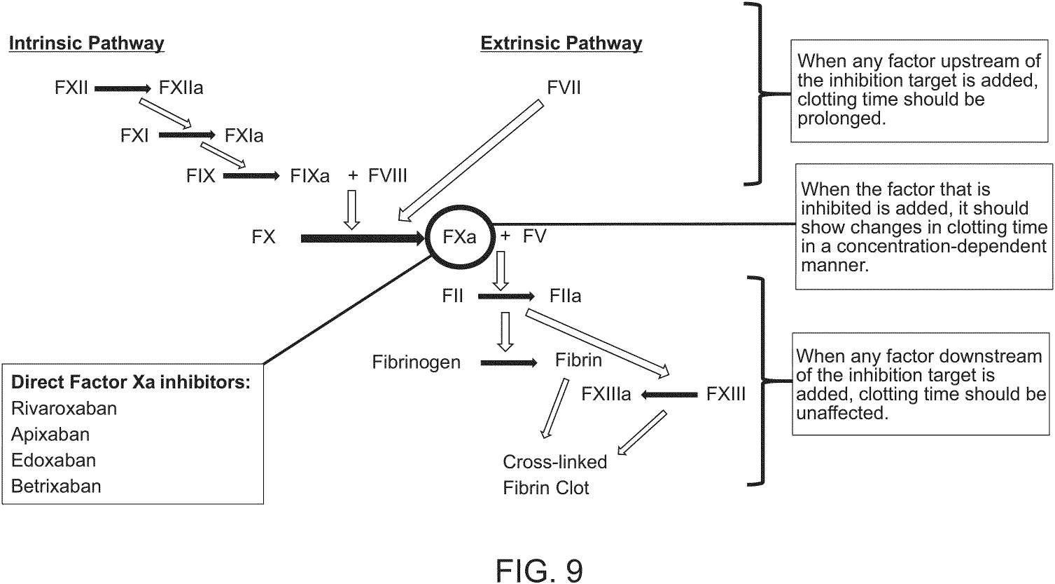

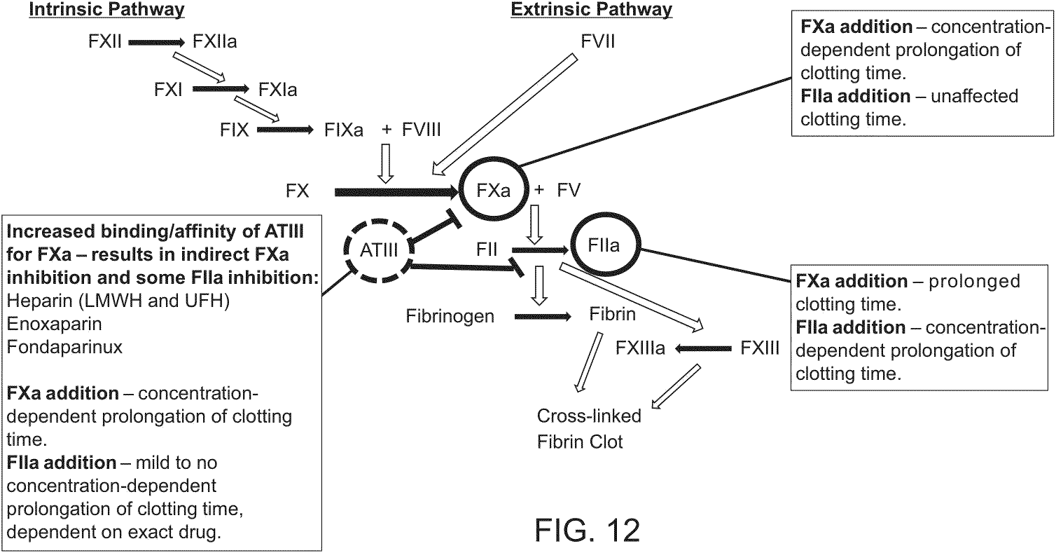

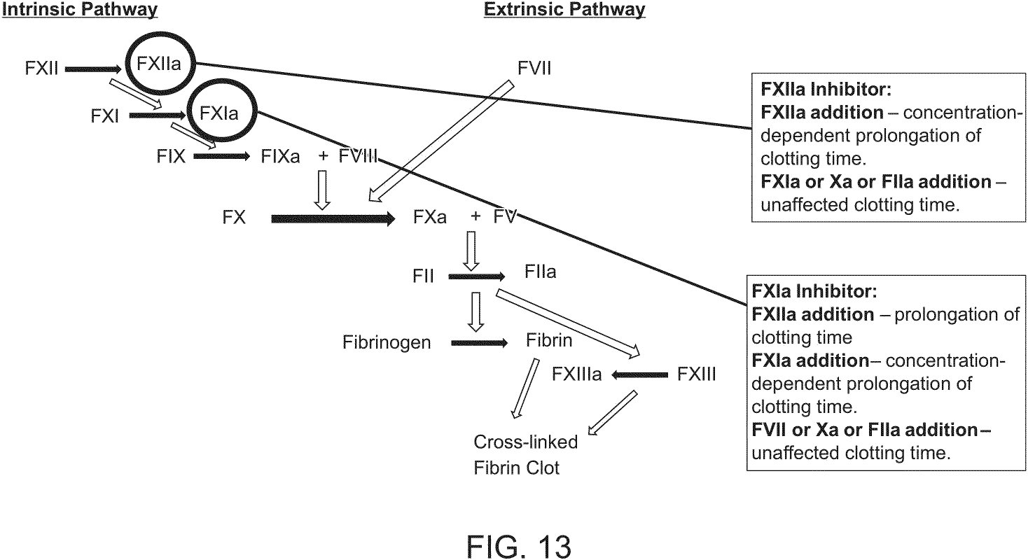

Examples of the utility of this assay include:
a) Detection of Factor IIa (thrombin) inhibitors and assessment of Factor IIa inhibition via the addition of Factor IIa at various concentrations (e.g., ranging from 10 µg/mL to 10 pg/mL; see, e.g., FIGS. 7D, 10, 11, 16).
b) Detection of Factor Xa inhibitors and assessment of Factor Xa inhibition via the addition of Factor Xa at various concentrations (e.g., ranging from 10 µg/mL to 10 pg/mL; see, e.g., FIGS. 3, 4A, 7A-7C, 9, 11, 16, 19-21).
c) Detection of Factor XI or XIa inhibitors and assessment of Factor XI or XIa inhibition via the addition of Factor XI or XIa and/or X or Xa at various concentrations (e.g., ranging from 10 µg/mL to 10 pg/mL; see, e.g., FIG. 13).
d) Detection of Factor XII or XIIa inhibitors and assessment of Factor XII or XIIa inhibition via the addition of Factor XII or XIIa and/or XI or XIa and/or X or Xa at various concentrations (e.g., ranging from 10 µg/mL to 10 pg/mL; see, e.g., FIG. 13).
e) Detection of all types of anticoagulant agents, including Heparin (fractionated, low molecular weight, or other) via the addition of Factor IIa, Xa, or a combination of the factors at various concentrations (e.g., ranging from 10 µg/mL to 10 pg/mL; see, e.g., FIGS. 4B and 12).
f) Detection and assessment of fibrinolytics (including, but not limited to, tissue plasminogen activator (tPA)) by the addition of various coagulation factors at various concentrations (e.g., ranging from 10 µg/mL to 1 pg/mL).
g) Detection of other coagulation abnormalities via the addition of an inhibited/abnormal/absent factor, including:
   i. Afibrinogenemia/dysfibrinogenemia via the addition of Fibrin
   ii. Factor V deficiency via the addition of Factor V and/or Va
   iii. Haemophilia A or B via the addition of Factor VIII and/or VIIIa, Factor IX and/or IXa
   iv. Von Willebrand factor disease via the addition of von Willebrand Factor
   v. Vitamin K-dependent abnormalities (warfarin, vitamin K deficiency, liver failure) via the addition of Factor II/VII/IX/X and/or IIa/VIIa/IXa/Xa
   vi. Antithrombin deficiency (kidney disease) via the addition of ATIII. See, e.g., FIGS. 9-13.

Embodiments of methods and devices described herein can be used to evaluate coagulation abnormalities (e.g., pro- or anti-thrombotic) using various coagulation detection technologies, such as those described herein, including: electrical impedance, the addition of beads and quantifying bead flow rate/number, measurement of flow velocity and/or pressure before and/or after the site of clot formation, thromboelastography, fluorescence detection (such as with fluorescent fibrinogen), turbidity, magnetic, flow dynamics (pressure or flow velocity), infrared light detection, infrared spectroscopy, detection using acoustic and/or photonic sensors, flow cytometry, and visual clotting detection.

Whole blood and plasma can be used in various embodiments.

Embodiments of the assays can be combined with ATP-luciferase assays in order to measure platelet and coagulation system function at the same time. This can provide evaluation of the coagulation cascade, as well as platelet function, via the degranulation of the platelet upon sufficient activation. Activation of the platelet can occur via the addition of the coagulation factors listed herein, or by the addition of specific platelet agonists, such as, e.g., adenosine diphosphate (ADP), adenosine triphosphate (ATP), epinephrine, collagen, thrombin, and ristocetin. This combined technique can be used to assess platelet function when patients are taking platelet inhibitors, such as aspirin or clopidogrel. These agonists can be added as a concentration gradient in combination with the coagulation factors. Luciferase is typically measured by light absorbance.

Coagulation abnormalities that can be detected or analyzed include, but are not limited to, congenital or hereditary coagulopathies and acquired coagulopathies.

Congenital or hereditary coagulopathies include acquired mutations and hereditary coagulopathies, i.e., inherited from a parent.

Congenital coagulopathies are present at birth and are likely due to a developmental abnormality that occurred in utero. Congenital coagulopathies may or may not be genetic. In some embodiments, the patient may have or be suspected to have a coagulation factor deficiency, which may be caused by the production of a deficient amount of the clotting factor, or the clotting factor is encoded by a gene with a mutation that decreases the function of the clotting factor.

Examples of congenital and hereditary coagulopathies include, but are not limited to:
a) Hemophilia A (Factor VIII deficiency)
b) Hemophilia B (Factor IX deficiency)
c) Hemophilia C (Factor XI deficiency)
d) Factor I (fibrinogen) deficiency
e) Factor V deficiency
f) Factor VII deficiency
g) Factor X deficiency
h) Factor XIII deficiency
i) Alpha2-antitrypsin deficiency
j) Alpha1-antitrypsin Pittsburgh (Anthithrombin III Pittsburgh) deficiency
k) Combined factor deficiencies (e.g., Factor V and VIII, Factor II, VII, IX, and X)
l) Platelet abnormalities (e.g., Gray platelet syndrome, Bernard-Soulier syndrome, von Willebrand disease, Glanzmann thrombasthenia, Hermansky-Pudlak syndrome, clopidogrel or aspirin resistance).

Causes of acquired coagulopathies include, but are not limited to: organ (e.g., liver) dysfunction or failure, bone marrow dysfunction or failure, trauma (e.g., automobile accident), surgery, infection (e.g., flavivirus, hemolytic uremic syndrome, sepsis, etc.), cancer, immobility, drugs (e.g., antibiotics, anticoagulation, fibrinolytics, thrombolytics, chemotherapy, fluids, etc.), neutraceuticals/pharmaceuticals, toxicities, envenomation (e.g., snake, spider, etc.), foods, auto-immune diseases (whether primary, acquired or idiopathic), implants (e.g., surgical), cardiovascular event(s) (e.g., a clot of blood anywhere in the body, including stroke, heart attack, etc.), vasculitis, transfusions (e.g., whole blood, packed red blood cells, plasma, platelets, etc.), transplants (e.g., bone marrow, kidney, liver, etc.), pregnancy (e.g., pre-eclampsia, eclampsia, diabetes, etc.), endocrine disease (e.g., pheochomocytoma, cushings, diabetes, etc.), chronic inflammatory disease (e.g., irritable bowel syndrome, irritable, bowel disease, colitis, etc.), disseminated intravascular coagulation, and infection.

Coagulopathies may also be iatrogenic (e.g., caused by medical treatment) or have idiopathic causes (e.g., cancer treatment, such as chemotherapy, or bone marrow transplant).

In some embodiments, the invention employs a microfluidic approach. The microfluidic device includes a series of channels in a substrate, each channel having an area with a geometry to trigger and/or localize formation of a clot, to allow for evaluation of clot formation in response to one or more reagents, such as the amount or concentration of an exogenously added coagulation factor. Each of the channels in the series has the same geometry, so as to trigger identical clot formation properties (when exposed to the same sample and reagents). By evaluating clot formation in the presence of a gradient of one or more coagulation factors, the invention allows for sensitive and specific detection of coagulation abnormalities or impairments, as described above.

Embodiments employing a microfluidic device, may involve the following procedures:
a) A sample is acquired from a patient;
b) One or more agonists (specific factor(s)) is/are added to the patient sample as described herein (either before entry into the microfluidic device or within the microfluidic device), each agonist at an increasing concentration across a series of channels in the microfluidic device;
c) +/- calcium is added if the sample is collected in an anticoagulant, such as sodium citrate or acid citrate dextrose;
d) The sample then flows through the microfluidic device where formation of a clot is triggered at a location within the channels;
e) The time to clot is measured and/or quantified at the location, and then recorded;
f) Multiple concentrations of the same agonist may be added to the aliquoted sample (in separate channels) to determine the presence and concentration of a coagulation cascade abnormality; concentrations can (but need not necessarily) range, for example, from about 0.75 ng/mL to about 750 ng/mL;
g) Multiple factors may be added to the aliquoted sample (in separate channels) to identify the part of the coagulation cascade that is functioning abnormally. By utilizing upstream and downstream factors, such as the use of Factor IIa and Xa in the identification of DOACs, one can identify the point at which normal clotting is recovered. Another example embodiment is identification of dysfibrinogenemia or afibrinogenemia: With a whole blood sample, one may have prolonged clotting times in the negative control lane (no agonist added); while addition of coagulation factors (such as Factors IIa and Xa) will not recover normal clotting times, the addition of fibrinogen to the sample recovers the clotting time since this missing/abnormal factor is being replaced in the device.

A microfluidic device for detecting coagulation can include plural channels formed in a substrate, each channel including a clot forming area having a geometry configured to trigger and/or localize formation of a clot. In some embodiments, the clot forming areas of the plural channels are arranged in a central region of the substrate. In some embodiments, the device further includes plural sample input ports, each sample input port connected to a first end of one of the plural channels. In some embodiments, the device comprises plural output ports, each output port connected to a second end of one of the plural channels. The input and output ports may be arranged in an alternating pattern at a periphery of the substrate. In some embodiments, the device comprises a common sample input port, in fluid connection with all channels or a series of channels.

A substrate can be, for example, any type of plastic, polydimethylsiloxane (PDMS), silicon, glass, or other material or combination of materials. In an embodiment, the device includes a substrate bound to glass, but other substrates can be used, such as glass on glass, PDMS on PDMS, silicon, any type of plastic, or combinations thereof. In one embodiment, the substrate is plastic. The substrate can be (but need not be) transparent to facilitate the detection of clot formation (vis-à-vis, e.g., imaging).

The device can include microfluidic channels with a diameter of about 50 µm, a height of about 11 µm, and a length of 100+ µm. Other channel dimensions can be employed.

One entry and one exit port for the sample input can be provided for each channel. Alternatively, devices can provide a single sample port for all channels or for one or more groups (or series) of channels.

In various embodiments, an agonist (e.g., a coagulation factor) is added to the sample prior to input into the device or the agonist is coated to, or otherwise pre-loaded within, the device prior to sample loading. In the case where one or more channels include the coagulation factor(s), the coagulation factor(s) may be in suspension, solution, or lyophilized, and may be surface-bound or not surface-bound. The coagulation factor(s) can be pre-included in the channel(s) (e.g., at the time of manufacturing the device), can be added prior to placing the sample into the device, or can be entered into the device through an input port (or multiple input ports) simultaneously with the sample or after the sample.

In an embodiment, calcium is added to the sample prior to input into the device. Calcium can be added within the device, through an additional port, or pre-loaded within the channel.

In an embodiment, 488-conjugated fibrinogen is added to the sample to detect the time it takes for a clot to form via the detection of cross-linking of the fibrinogen.

In bright-field, clot formation can also be detected by visualizing the cross-linking of fibrin and by the stopping of the flow of the sample through the microfluidic channel, which can be performed with or without an additional flushing step to flush out material not associated with a clot.

In an embodiment, the sample is loaded into the device or microfluidic cartridge via capillary action. The sample can also be forced to flow through the channel, e.g., through the use of a vacuum, syringe-pump, or other suitable means, including, in some embodiments, gravity. The sample can also be encouraged to load by capillary action or flow by using coating that alters the surface properties of the microfluidic device (e.g., substrate), such as by making it hydrophilic.

In an embodiment, the design of the microfluidic channel(s) includes one area of an altered geometry (including different angled bends and/or diameters) in order to create one area of flow separation and stasis to trigger and/or localize formation of the blood or fibrin clot. The time that it takes for the clot to form can be quantified and recorded.

In an embodiment, the device is used to detect the presence and assess the effect of anticoagulation agents, e.g., FXa inhibitors, FIIa inhibitors, heparin, and vitamin K antagonists (e.g., warfarin) by assessing the time is takes to form a clot.

The measured clot formation time is correlated to the amount of clotting inhibition that is resultant from an anticoagulant in the sample. This process can also be applied to a fibrinolytic drug. This process can also be applied to other pathologies, including acquired or congenital causes of abnormal clotting times, as described herein.

In an embodiment, the device provides a read-out in a relatively short period of time, for example, in about 3-10 minutes, and, in a particular example, in about 5 minutes.

Example microfluidic devices and assays are described below and illustrated in the figures.

### EXAMPLES

### Example 1

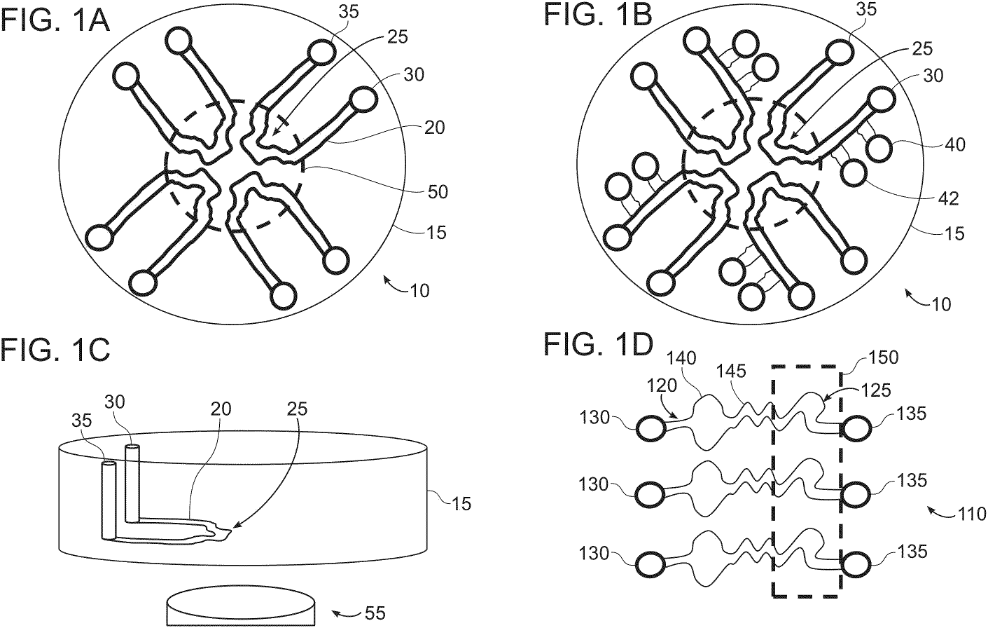

FIGS. 1A-1D are schematic illustrations of microfluidic device layouts according to example embodiments of the invention.

FIG. 1A is a top view of a circular layout (it can also be any symmetrical polygon with a center point) of microfluidic device 10 having one or more continuous microfluidic channels (e.g., microchannels) 20 formed in a substrate 15, each channel connected to one inlet (input port) 30 and one outlet (output port) 35. A portion of the channel, e.g., the center of the channel, can have a unique shape, e.g., a clot forming/localizing area 25, in order to result in flow separation or disruption, or stasis of sample flow to promote clot formation. There may be two or more of these microfluidic channels in this single device, dependent on the specific assay being used. This design can allow for multiple samples, such as three or more samples, e.g., up to 10 samples, or more than 10 samples, to be evaluated simultaneously. Typically, each sample (or each aliquot of a sample) requires a separate channel. In FIG. 1A, four channels are illustrated, each having a clot forming/localizing area 25 located proximally on the microfluidic device, e.g., locate in a central region of the microfluidic device. The sample can enter the device through the inlet manually or by an electronic dispenser and will go through the microfluidic channel by an applied pressure/vacuum, capillary action, or via chemical interactions, such as if the microfluidic channel is coated with or made of a hydrophilic material. In this example set-up, the agonists +/- calcium +/- clot detection reagents must be added to the main inlet, pre-mixed into the sample, or must be coated on to the inlet or the microfluidic channel. (The term “+/-”, as used herein means “with or without.”) All of the clot forming/localizing areas may be viewed in one single imaging field (dashed circle 50 encompassing clot forming areas 25) at magnification that may range from, for example, 2X-10X.

FIG. 1B is a top view of a similar layout as in FIG. 1A but with examples of multiple inlet ports 30, 40, 42 for each channel 20. This allows for the agonist +/- calcium +/- clot detection reagents to be added to the sample within the microfluidic channel. There can be one or more additional inputs 40, 42 and they can individually connect directly to the main channel 20 or the main input area, or some may connect indirectly to each other with at least one connecting to the main channel or the primary input port.

FIG. 1C is a side view of a microfluidic device layout illustrating input 30 and output 35 ports of a channel 20 in substrate 15. Only one channel is shown, but one or more channels may be provided as illustrated in FIG. 1A. In addition, one or more input ports may be provided for each channel, as illustrated in FIG. 1B. As schematically illustrated in FIG. 1C, a detection device 55 can be provided to measure clot formation in each of the channels. The detection device 55 can include an imaging sensor to detect clot formation, e.g., clot formation times. Imaging can be bright-field imaging as described herein. The detection device may use any of the other measuring/detection methodologies described herein.

FIG. 1D is a top view of a microfluidic device 110 having an alternate layout that may be utilized for various assays. There can be one or more inlets (input ports) 130 with one outlet (output port) 135 per sample input and channel 120. An area of shape change 125 to stimulate clot formation is included in each channel 120. The channels are arranged in a parallel fashion in order to allow for visualization of the clot formation/localization areas 125 within one field of view (dashed rectangle 150) at magnification that may range, for example, from 2X-10X. Each channel can include one or more areas 140 for agonist and/or calcium addition and a region 145 for mixing. In the example shown, the channels 120 have identical geometries.

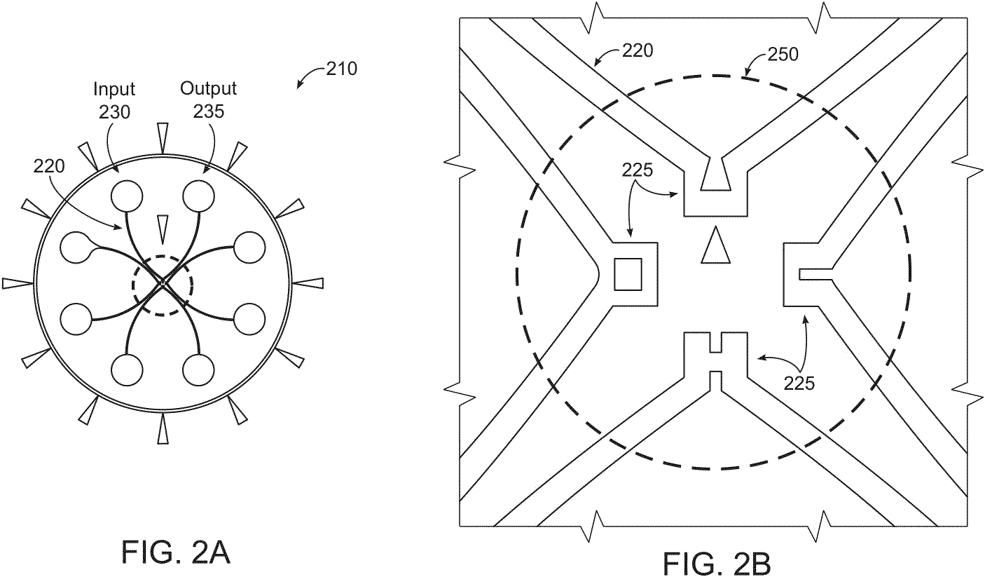

FIGS. 2A and 2B illustrate a circular microfluidic clotting device 210 according to an example embodiment. As shown, the device includes four channels 220, each channel including a clot forming/localizing area 225 having a geometry to trigger and/or localize clot formation. The clot forming areas 225 are arranged in a central region. Each channel 220 is connected to an input port 230 and an output port 235. The input and output ports of all the channels are arranged in an alternating pattern at a periphery of the device 210. The dashed circle 250 in the center indicates a general field of view encompassing ‘clotting areas’ 225 of all input channels. The configuration of channels shown in FIG. 2A is a configuration in which wicking capillary flow occurs, but many other configurations are possible. A particular configuration may be selected based on one or more criteria, such as whether the configuration is particular advantageous for manufacturing the device.

FIG. 2B is a magnified view of the central portion of the device 210 of FIG. 2A illustrating examples of clot forming/localizing areas 225 within the field of view. The clot forming areas can have configurations conducive to formation of a clot that can be quantified. The clot forming areas can have shapes designed to cause flow separation, stasis, flow disturbances, or combinations thereof, for clot formation, and may have shapes designed to cause flow disturbance for clot formation. In the example, the clot forming areas have different shapes to illustrate various shapes that can be used. Typically, the shapes will be the same for each channel so as to ensure the same flow conditions in each channel. The shapes of clot forming areas illustrated in FIG. 2B are examples and not all-inclusive of the shape variations that can be used.

As illustrated in FIG. 2B, each clot forming area can be configured (e.g., shaped) such that a sample flowing through a clot forming area is forced to change direction at least once, preferably multiple times. Each change in direction can be in the range of, for example, about 45 degrees to about 135 degrees, of about 60 degrees to about 120 degrees, of about 75 degrees to about 105 degrees, or of about 90 degrees. In addition, one or more flow disruptors, such as protrusions or islands, can be provided to disrupt flow. As a sample passes through the clot forming area, it encounters flow disruptor(s) and is forced to flow around the disruptor(s). A disruptor may include corners or pointed edges, and can be triangular, rectangular, or otherwise shaped as illustrated in FIG. 2B. A combination of disruptors and other structural features, or just other structural features, may form a circulatory region, where sample flow in a circular pattern interacts with new sample entering the region as other sample departs. Eddy currents behind disruptors, from a fluid flow point of view, may also encourage coagulation as sample interacts with other sample at intersections (e.g., turbulence intersections) of fluid flow and sample in an eddy region.

In some embodiments, the disruptor can include a concavity (e.g., FIG. 3A). A clot forming/localizing area may include a narrowing of the channel. By changing the direction of sample flow and/or changes in diameter, angle, and/or shape of the channel, and/or forcing the sample to flow around one or more disruptors, the clot forming areas introduce flow separation and stasis of sample flow to promote clot formation. Typically, the channels and clot forming areas are arranged in a symmetrical pattern in order to provide the same flow characteristic for each of the channels.

### Example 2

A general protocol for performing the assay according to an embodiment of the invention is as follows:
a) Add together sample, agonist, +/- calcium, +/- clot detection agent
   i. Calcium to a final concentration of 0.2 mM (This concentration is particularly suitable for use with 3.2% buffered sodium citrate. If another anticoagulant is used, the concentration of calcium may not be 0.2 mM.)
   ii. Clot detection agents can include fluorescent labeled fibrinogen, magnets, beads (may be fluorescent or colored)
b) Load into microfluidic device
   i. See, e.g., FIGS. 1A-1D, 2A and 2B for examples of input loading configuration and order
c) Temperature control
   i. Room temperature
   ii. May increase up to 37° C. (body temperature) (Body temperature is typically 37° C. but the temperature of the assay run can be changed according to the patient’s actual temperature. For example, if a patient has a fever, the temperature of the assay run can be increased.)
d) Perform clot detection and measure time of clot formation (e.g., 4-12 minutes)
e) Log time when each sample starts to form a clot

### Example 3

FIGS. 3A-3C illustrates clot detection using plasma and fluorescent-labeled fibrinogen with a microfluidic device 310 having four channels 320 with clot forming/localizing areas 225 according to an example embodiment. The microfluidic device is similar to the device show in FIGS. 2A and 2B except that all clot forming areas 325 have the same shape. Each clot forming/localizing area 325 includes a protrusion to disrupt sample flow. In this example, as shown in FIG. 3A, the protrusion generally is triangular in shape. Two sides of the protrusion are straight and one side is concave. Each clot forming area 325 causes the flow to change direction four times, including two 90 degree changes in direction.

In an example, the process of clot detection can include the following procedural steps:
a) A plasma sample is pre-mixed to include: 6 µL plasma + 0.6 µL agonist (10% volume to sample) + 0.6 µL Calcium (stock 2 mM, 10% volume to sample) + 0.6 µL Fibrinogen (this can vary in concentration, in general <10% volume of sample). The foregoing values can be adjusted and changed and similar results obtained.
b) For each channel, an aliquot of the pre-mixed sample is placed into the input port of the channel.
c) The sample aliquot is drawn into the channel by capillary action.
d) The channels are imaged for 10 minutes at 37° C., and the time to detect a clot is recorded.

The example in FIG. 3B shows a fluorescent image taken of the microfluidic channels at one time point (5 minutes). The plasma sample used contains 250 ng/mL of Apixaban. An agonist, Factor Xa (FXa) at various concentrations (0.75 ng/mL FXa, 7.5 ng/mL FXa, and 75 ng/mL FXa) or buffer alone (negative control) was added to the plasma sample, along with calcium and 488-conjugated fibrinogen. Crosslinking of the fluorescent fibrinogen is indicative of the formation and presence of a cross-linked fibrin clot. Higher concentrations of the FXa (7.5 ng/mL FXa, and 75 ng/mL FXa), visible in the channels on the right in FIG. 3B, result in clot formation earlier than the lower concentration (0.75 ng/mL FXa) or the negative control, visible in the channels on the left in FIG. 3B. FIG. 3C is a magnified view of a clot forming area of one channel illustrating a cross-linked fibrin clot.

### Example 4

FIGS. 4A and 4B are fluorescent images illustrating clot detection using whole blood in a parallel microfluidic channel device 410 according to an example embodiment. Microfluidic channels 420 were pre-coated with agonist, Factor Xa, at various concentrations (7.5 ng/mL, 75 ng/mL, 750 ng/mL) or with buffer alone (negative control). The fluorescent images are taken at one time point (10 minutes). Microfluidic channels were washed with buffer prior to use to leave only bound FXa within the microfluidic channel. Fresh whole blood was placed into each input port and the blood was drawn in through capillary action. The blood was left to flow for 10 minutes and then the channel was gently washed with buffer. Depicted is a brightfield image of two samples evaluated. The sample in FIG. 4A contained no anticoagulant (finger prick of blood), which resulted in clots in all 4 channels, including the negative control. The sample in FIG. 4B contained unfractionated heparin (which was added to the finger prick of blood), which resulted in a gradient of clot formation dependent on the concentration of FXa in the channel. Almost no cells were adhered in the negative control, indicating minimal clot formation. Unfractionated heparin inhibits Factors IIa and Xa in an antithrombin III-dependent fashion, which is why the addition of these factors at appropriate concentrations can help recover the clotting capability of the sample.

### Example 5

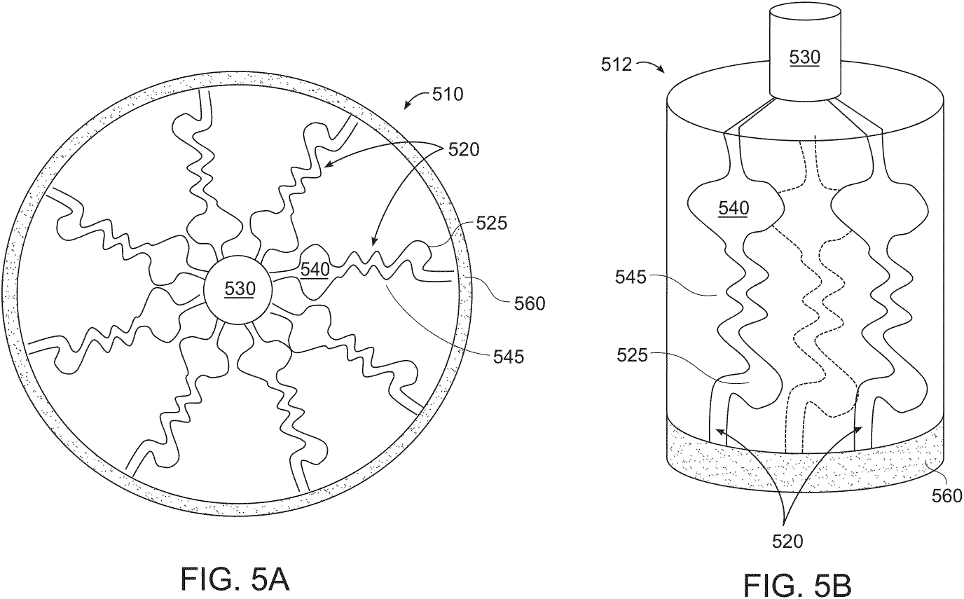

FIGS. 5A and 5B illustrate additional embodiments of microfluidic device designs that include the features of: (1) each channel subjects the blood/plasma to equal conditions and (2) there is a clot-promoting geometry within each channel where clotting detection is optimized and performed. FIG. 5A illustrates a device 510 including circular array of symmetrical channels 520 surrounding and connected to a single sample input 530, where each channel has a clot-promoting and/or localizing area 525. The channels 520 may or may not also include one or more areas for agonist and/or calcium addition 540 and/or mixing 545. FIG. 5B illustrates an alternative embodiment of a device 512 utilizing a cylindrical design with a single sample input port 530 that divides into multiple symmetrical channels 520 with a clot-forming area 525 with or without an area for agonist/calcium 540 addition and/or mixing 545. Both devices 510, 512 may also include a sample collection reservoir 560 with or without an absorbent filter.

### Example 6

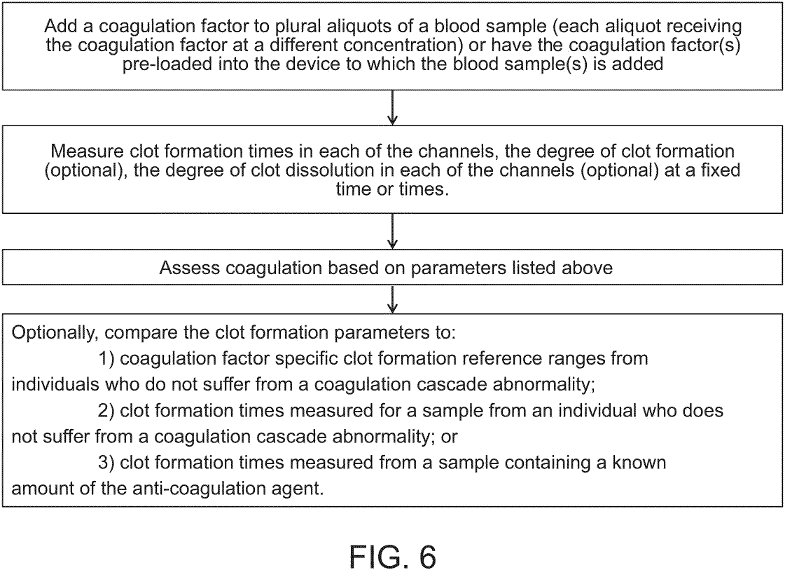

FIG. 6 is a flow diagram of a method of assessing coagulation in a blood sample according to example embodiments of the invention. The blood sample can be a whole blood sample or a plasma sample. According to the method, a coagulation factor is added to plural aliquots of the blood sample. Each aliquot can receive the coagulation factor at a different concentration. The plural aliquots can be applied to plural channels of a microfluidic device. Alternatively, or in addition, the coagulation factor(s) can be pre-coated on or into the device to which the blood sample is applied. Clot formation times are measured in each of the channels and coagulation is assessed based on the clot formation times measured. Alternatively, or in addition, degree of clot formation (optionally, degree of clot dissolution) in each of the channels is measured at a fixed time or times, and coagulation is assessed based on the degree of clot formation (optionally, degree of clot dissolution) measured.

Optionally, as illustrated in FIG. 6, the clot formation times can be compared to a reference value or reference ranges. In one example, the clot formation times are compared to coagulation factor specific clot formation reference ranges from individuals who do not suffer from a coagulation cascade abnormality. This is useful, e.g., to detect a coagulation cascade abnormality in the blood sample. In another example, the clot formation times are compared to clot formation times measured for a sample from an individual who does not suffer from a coagulation cascade abnormality. This is also useful, e.g., to detect a coagulation cascade abnormality in the blood sample. In yet another example, the clot formation times are compared to clot formation times measured for a sample containing a known amount of an anticoagulation agent. This is useful, e.g., to detect the anticoagulation agent in the blood sample.

The microfluidic device for use in the method of FIG. 6 can be any microfluidic device described herein having plural channels, such as the devices illustrated in FIGS. 1A-1D, 2A-2B, 3A-3C, 4A-4B and 5A-5B. In an embodiment, the device includes plural channels formed in a substrate, each channel including a clot forming area having a geometry configured to trigger and/or localize formation of a clot, the clot forming areas of the plural channels being arranged in a central region of the substrate; plural input ports, each input port connected to a first end of one of the plural channels; and plural output ports, each output port connected to a second end of one of the plural channels, the input and output ports being arranged in an alternating pattern at a periphery of the substrate.

### Example 7

FIGS. 7A-7D illustrate example clotting curves for various FXa and FIIa inhibitors at various concentrations. The time it takes for each of the combinations to form a clot is then plotted. The clotting curve for each concentration of inhibitor is dependent on the presence and concentration of the anticoagulant in the sample. The figures illustrate the time-to-clot for four (4) different DOACs when exposed to agonists at various concentrations. The time-to-clot increases as the concentration of the inhibitor increases, demonstrating an increase in functional anticoagulation. Concentration of the agonist (FXa for FIGS. 7A-7C, and FIIa for FIG. 7D) is plotted on the X-axis for each of the figures.

FIG. 7A is a graph of example data illustrating detection of Rivaroxaban. The graph shows clotting curves for different concentrations of the inhibitor Rivaroxaban (0 ng/mL, 250 ng/mL, and 500 ng/mL). Each curve shows average clot detection time (minutes; y-axis) as a function of agonist (FXa) concentration (ng/mL; x-axis). The data shown in the graph can be summarized as follows:

At a concentration of 0 ng/mL Rivaroxaban, clot formation detected in < 2.5 minutes with agonist concentration down to 7.5 ng/mL.

At a concentration of 250 ng/mL Rivaroxaban, clot formation time is significantly longer than the negative control but lower than 500 ng/mL with agonist concentration down to 375 ng/mL.

At a concentration of 500 ng/mL Rivaroxaban, clot formation detected < 2.5 minutes down to 750 ng/mL.

FIG. 7B is a graph of example data illustrating detection of Apixaban. The graph shows clotting curves for different concentrations of Apixaban (0 ng/mL, 250 ng/mL, and 500 ng/mL). As in FIG. 7A, each curve shows average clot detection time (minutes; y-axis) as a function of agonist (FXa) concentration (ng/mL; x-axis). The data shown in the graph can be summarized as follows:

At a concentration of 0 ng/mL Apixaban, clot formation detected in < 2.5 minutes with agonist concentration down to 7.5 ng/mL.

At a concentration of 250 ng/mL Apixaban, clot formation detected in < 2.5 minutes with agonist concentration down to 75 ng/mL.

At a concentration of 500 ng/mL Apixaban, clot formation detected in < 2.5 minutes with agonist concentration down to 938 ng/mL.

FIG. 7C is a graph of example data illustrating detection of Edoxaban. The graph shows clotting curves for different concentrations of Edoxaban (0 ng/mL, 250 ng/mL, and 500 ng/mL). As in FIG. 7A, each curve shows average clot detection time (minutes; y-axis) as a function of agonist (FXa) concentration (ng/mL; x-axis).

FIG. 7D is a graph of example data illustrating detection of Dabigatran. As in FIGS. 7A and 7B, the graph of FIG. 7D shows clotting curves for different concentrations of the inhibitor, here Dabigatran (0 ng/mL, 25 ng/mL, 250 ng/mL, and 500 ng/mL). Each curve shows average clot detection time (minutes; y-axis) as a function of agonist (FIIa) concentration (ng/mL; x-axis). The data shown in the graph of FIG. 7D can be summarized as follows:

At a concentration of < 25 ng/mL Dabigatran, clot formation detected in < 2.5 minutes with agonist concentration down to 71 ng/mL.

At a concentration of 250 ng/mL Dabigatran, get clot formation detected in < 2.5 minutes with agonist concentration down to 710 ng/mL.

At a concentration of 500 ng/mL Dabigatran, clot formation detected in < 2.5 minutes down to 710 ng/mL.

Automation can be employed to reduce variation between samples and assays.

### Example 8

In addition to the detection of the presence of FXa inhibitors and estimation of their relative concentrations, the assay described here can differentiate FXa inhibitors from FIIa inhibitors by selecting appropriate upstream and downstream clotting factors to add to the samples.

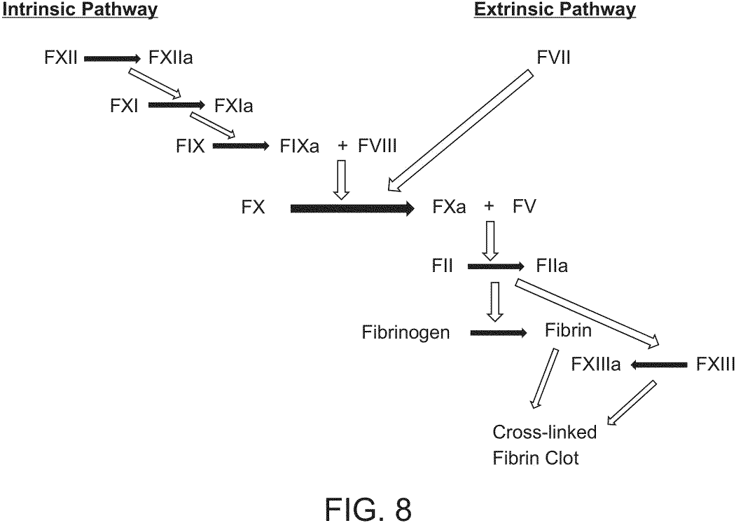

FIG. 8 illustrates a basic clotting cascade that can guide the selection of appropriate clotting factors, as further described in the following examples. As shown in FIG. 8, the cascade includes an intrinsic pathway and an extrinsic pathway, both of which can lead, via a common pathway of the cascade, to a cross-linked Fibrin clot. The intrinsic pathway can, for example, be activated by surface contact. The extrinsic pathway can be activated, for example, by tissue trauma.

### Example 9

FIG. 9 is a schematic diagram providing a demonstration of how to detect Factor Xa (FXa) inhibition/deficiency/abnormality of function. The addition of upstream (not active or activated) coagulation factors, including but not limited to FXII, FXI, FIX, FVIII, will demonstrate prolongation of clotting time, e.g., as compared to addition of a downstream factor. Alternatively, prolongation of clotting time can be determined with reference to a control clotting time. However, the addition of downstream (not active or activated) coagulation factors, including but not limited to FII, FI, will demonstrate unaffected (e.g., normal) clotting time, which can serve as a control. The addition of FXa will demonstrate prolongation of clotting time in a concentration-dependent manner, and even at high concentration of the upstream factor, the clotting team will likely not reach the control. As illustrated, example direct FXa inhibitors include Rivaroxaban, Apixaban, Edoxaban, and Betrixaban.

### Example 10

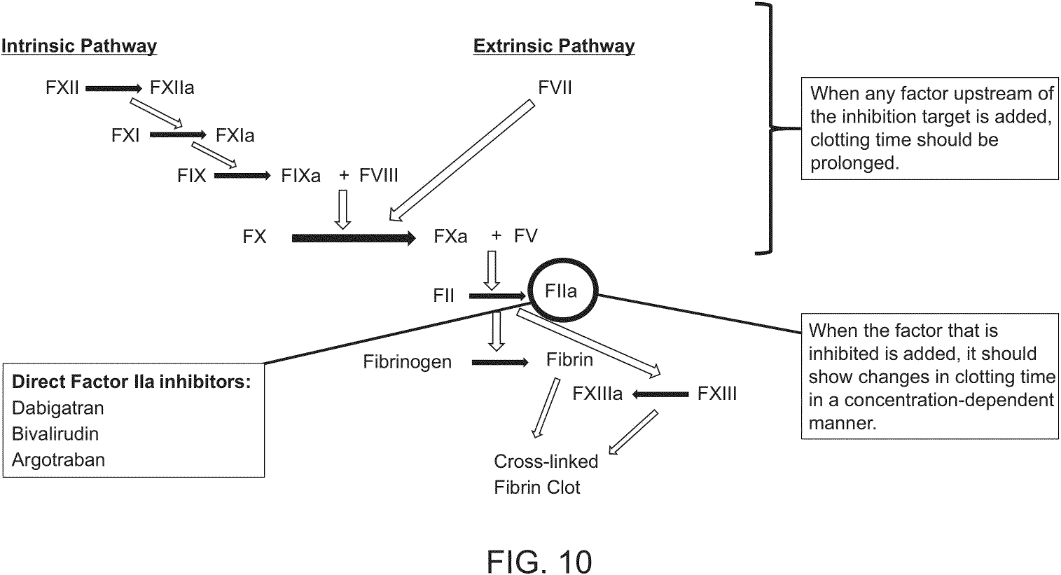

FIG. 10 is a schematic diagram providing a demonstration of how to detect Factor IIa (FIIa) inhibition/deficiency/abnormality of function. The addition of upstream (not active or activated) coagulation factors, including but not limited to FXII, FXI, FIX, FX, FV, FVIII, will demonstrate prolongation of clotting time. The addition of downstream (not active or activated) coagulation factors, including but not limited to FI, will demonstrate unaffected clotting time. The addition of FIIa will demonstrate prolongation of clotting time in a concentration-dependent manner. As illustrated, example direct FIIa inhibitors include Dabigatran, Bivalirudin, and Argotraban.

### Example 11

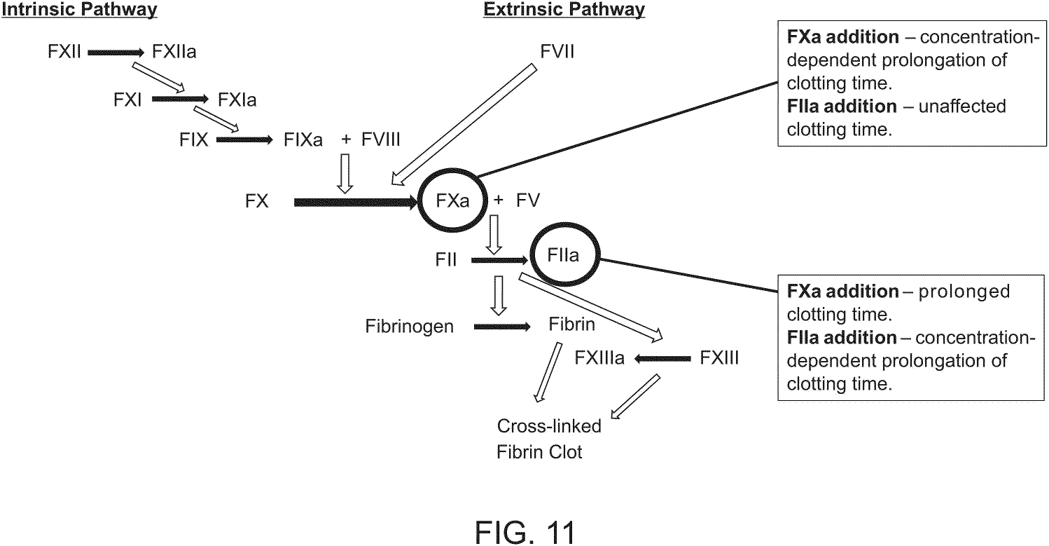

FIG. 11 is a schematic diagram providing a demonstration of how to detect and differentiate between FIIa and FXa inhibition in a sample. In the presence of a FXa and FIIa inhibitor, addition of upstream (not active or activated) coagulation factors, including but not limited to FXII, FXI, FIX, FVIII, will demonstrate prolongation of clotting time. The addition of FXa to the sample will demonstrate prolongation of clotting time, in a concentration-dependent manner for FXa and FIIa inhibition. The addition of FIIa to the sample will demonstrate prolongation of clotting time, in a concentration-dependent manner in the presence of FIIa inhibition but will demonstrate unaffected clotting time in the presence of FXa inhibition.

### Example 12

FIG. 12 is a schematic diagram providing a demonstration of how to detect indirect FXa inhibition/deficiency/abnormality of function. The addition of upstream (not active or activated) coagulation factors, including but not limited to FXII, FXI, FIX, FVIII, will demonstrate prolongation of clotting time. The addition of downstream (not active or activated) coagulation factors, including but not limited to FII, FI, will demonstrate normal clotting time or slight prolongation of clotting time in a concentration-dependent manner dependent on the type of inhibitor present. The addition of FXa will demonstrate prolongation of clotting time in a concentration-dependent manner. This is due to secondary FXa inhibition via the presence of a drug that increases the affinity/binding of Antithrombin III (ATIII) to FXa, thereby inhibiting it. Embodiments can include detection of ATIII, thereby detecting indirect inhibition of FXa, FIIa, or both. Drugs that increase binding/affinity of ATIII for FXa include Heparin, e.g., Low Molecular Weight Heparin (LMWH) and Unfractionated Heparin (UFH), Enoxaparin, and Fondaparinux.

### Example 13

FIG. 13 is a schematic diagram providing a demonstration of how to detect and differentiate between FXIIa and FXIa inhibition in a sample. In the presence of a FXIIa inhibitor, addition of FXIIa will result in concentration-dependent prolongation of clotting time. The addition of factors added downstream, including but not limited to, FXI, FIX, FVIII, FX, FII, FV, would result unaffected clotting times. In the presence of a FXIa inhibitor, addition of FXIIa will result in prolongation of the clotting time. Addition of FXIa would result in a concentration-dependent prolongation of clotting time. The addition of factors added downstream, including but not limited to, FIX, FVIII, FX, FII, FV, would result unaffected clotting times. This approach can also be used in various combinations to perform a comprehensive panel for the detection and differentiation of FXIIa inhibitors, FXIa inhibitors, FXa inhibitor, and FIIa inhibitors.

### Example 14

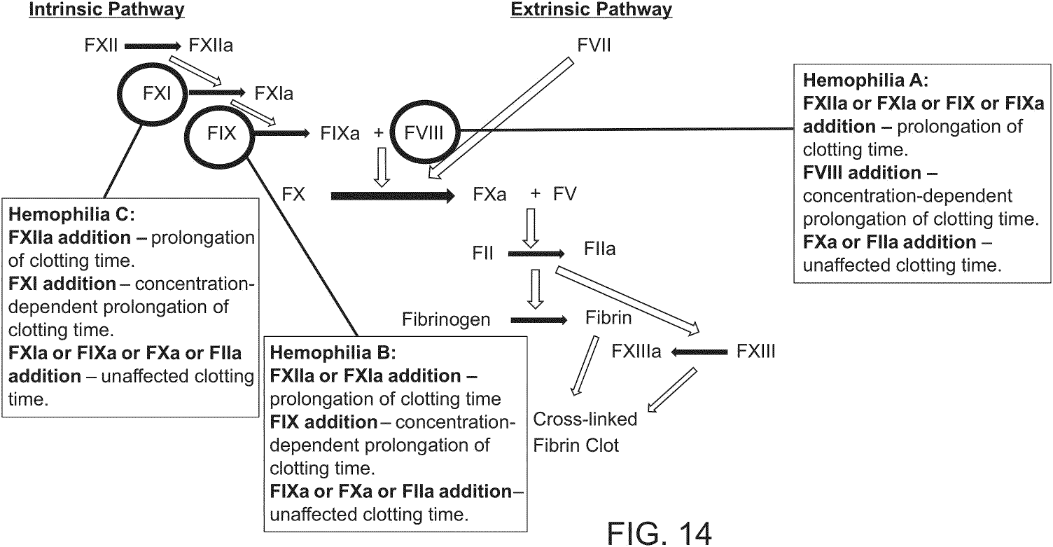

FIG. 14 is a schematic diagram providing a demonstration of how to detect and differentiate between various types of hemophilia. Hemophilia C would result in prolongation of clotting time with the addition of FXIIa, concentration-dependent prolongation with the addition of FXI, and unaffected clotting times with the addition of FXIa or any other downstream factor. Hemophilia B would result in prolongation of clotting time with the addition of FXIIa and FXIa, concentration-dependent prolongation with the addition of FIX, and unaffected clotting times with the addition of FIXa or any other downstream factor. Hemophilia A would result in prolongation of clotting time with the addition of FXIIa, FXIa, concentration-dependent prolongation with the addition of FVIII, and unaffected clotting times with the addition of FXa or any other downstream factor.

For congenital disorders, embodiments can add non-activated factor(s) for detection, whereas non-activated factor(s) can serve as control.

### Example 15

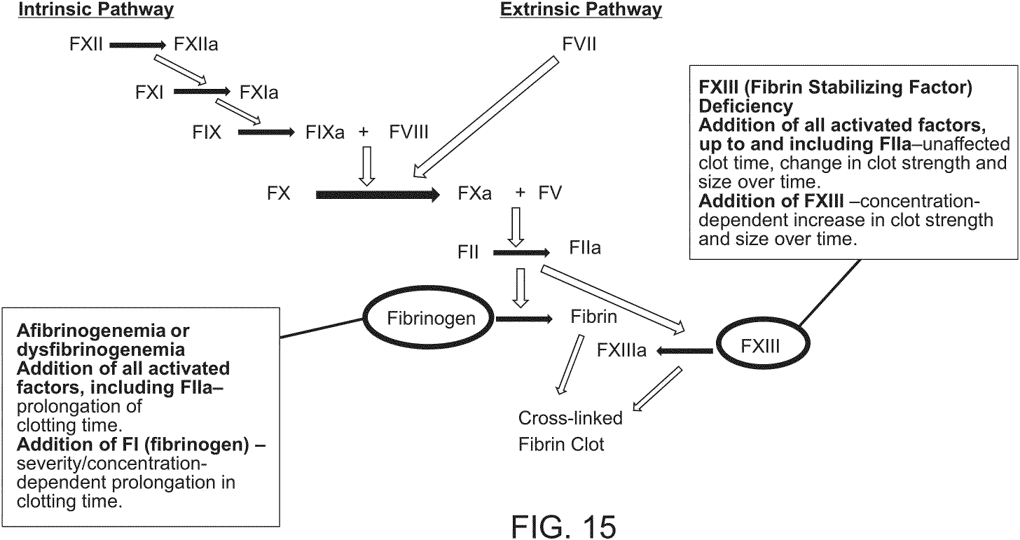

FIG. 15 is a schematic diagram providing a demonstration of how to detect problems with fibrinogen, i.e., Factor I (FI), or FXIII. Afibrinogenemia or dysfibrinogenemia would result in prolongation of clotting time with the addition of all factors upstream of FI, concentration-dependent prolongation with the addition of FI. FXIII deficiency/abnormality would result in changes in clot strength and clot stability over time with the addition of all factors upstream of FXIII, concentration-dependent changes in clot strength and stability of time with the addition of FXIII.

### Example 16

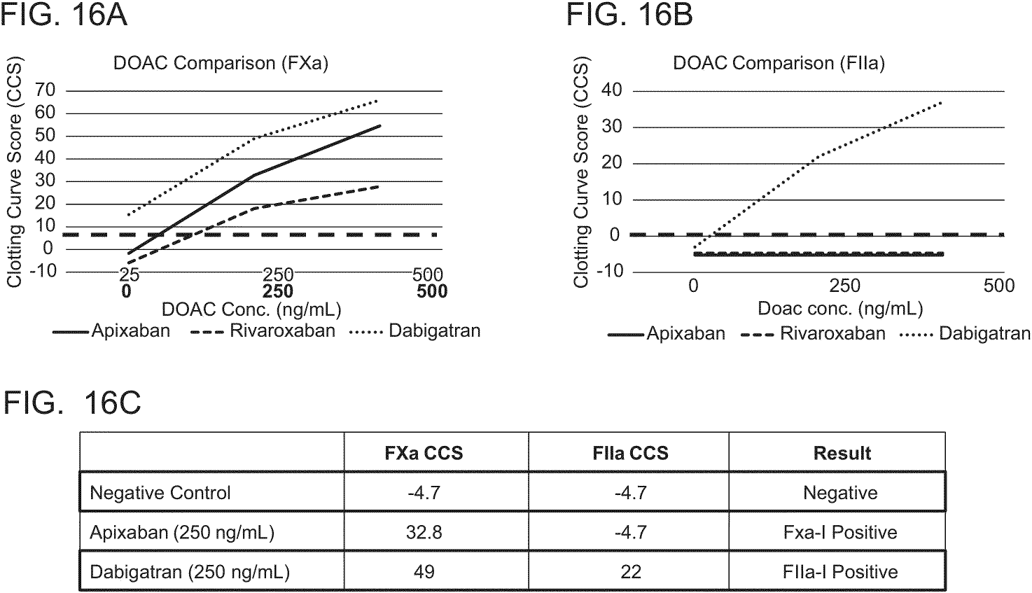

FIGS. 16A-16C illustrate Clotting Curve Scores (CCS) for FXa and FIIa inhibitors at various concentrations. Raw data of the clotting times of each of the agonists at various concentrations are used to calculate a single Clotting Curve Score (CCS) based on multivariate statistical modeling. This CCS can then be used as a single whole number to bin patients into positive or negative for specific inhibitors. This CCS can also be used to extrapolate the functional concentration of the drug in the patient sample. Functional concentration represents the amount of anticoagulation secondary to the drug in the blood sample. FIG. 16A shows how the CCS of two FXa inhibitors (Apixaban, Rivaroxaban) and one FIIa inhibitor (Dabigatran) vary dependent on concentration using FXa as the agonist. FIG. 16B shows how the CCS of the two FXa inhibitors and the one FIIa inhibitor vary dependent on concentration using FIIa as the agonist. FIG. 16C demonstrates how the CCS for each agonist can be used to identify the type of inhibitor in the sample.

### Example 17

FIG. 17 shows Table 1 that provides patient descriptive statistics. Citrated plasma samples were collected from patients admitted into the Massachusetts General Hospital Emergency Department. All plasma samples had clinician-ordered coagulation tests (PT/INR, aPTT, DTT, or other). Patient samples were evaluated using an embodiment of the assay described herein. Patient medical records were reviewed for the administration of history of anticoagulants. All patient samples were collected following Institutional Review Board (IRB) approval and regulations at both the Massachusetts General Hospital and the Massachusetts Institute of Technology.

### Example 18

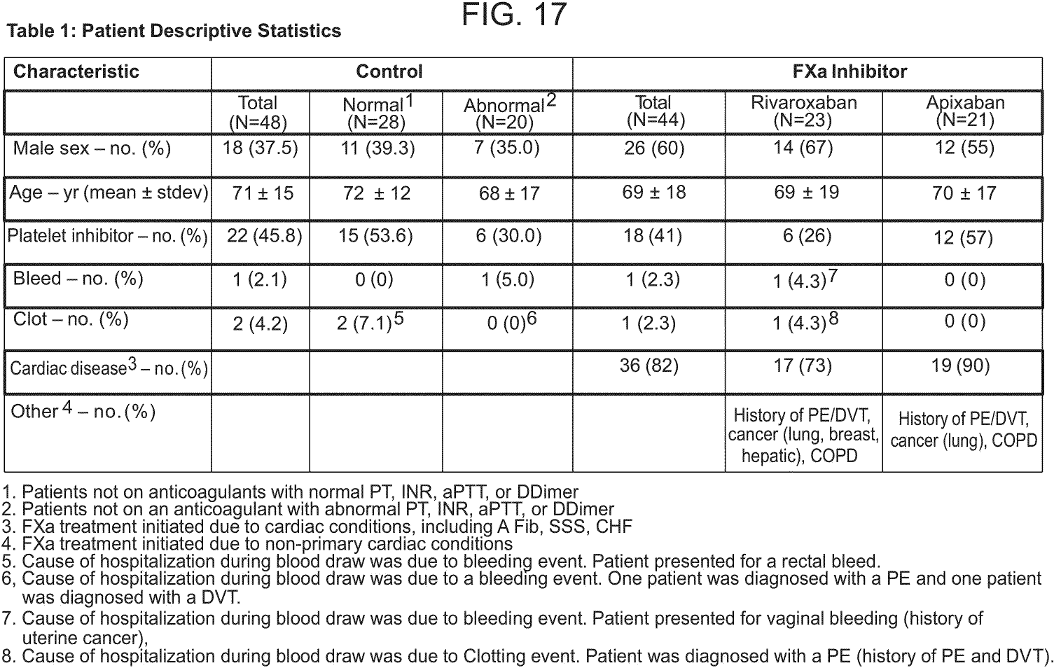

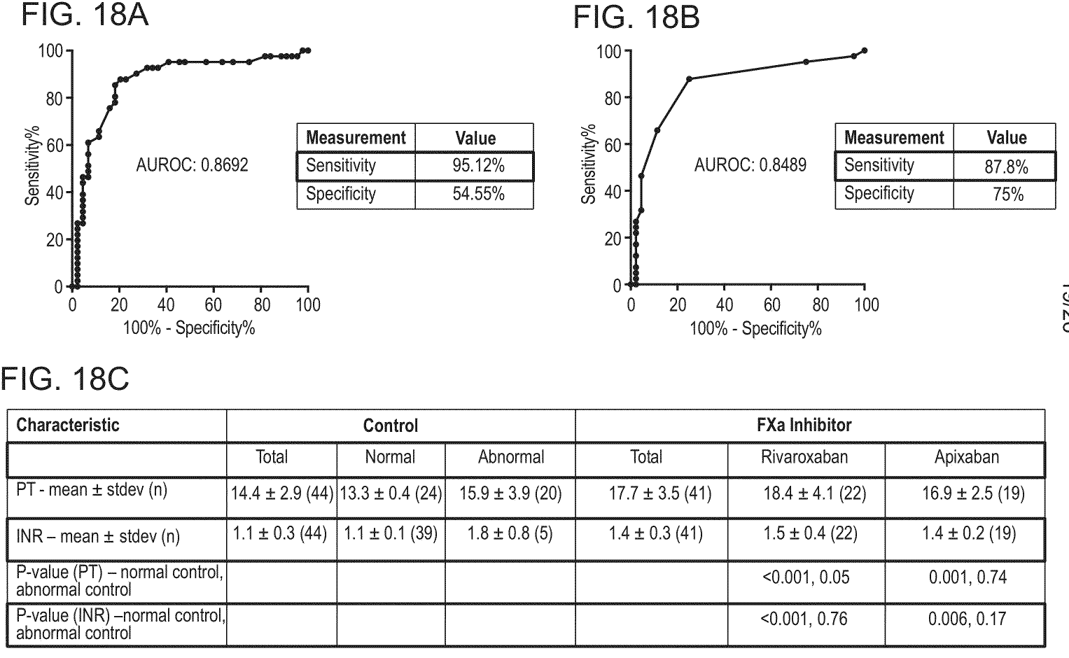

FIGS. 18A-18C illustrate prothrombin time (PT) and international normalized ratio (INR) is sensitive but not specific for FXa-I anticoagulation. Both PT and INR were compared between control patients and patients documented to be on FXa-I. Abnormal PT was defined as >14 seconds and abnormal INR was defined as >1.2. FIGS. 18A and 18B show ROC curves comparing PT and INR of total controls to patients on FXa-I. FIG. 18C shows a table of descriptive statistics of patients with PT and INR results evaluated. One-way ANOVA was used to compare normal and abnormal controls with both rivaroxaban and apixaban. Significance was defined as p < 0.05. Results show that, when compared to abnormal controls, there is no significance compared to the FXa-I patients.

### Example 19

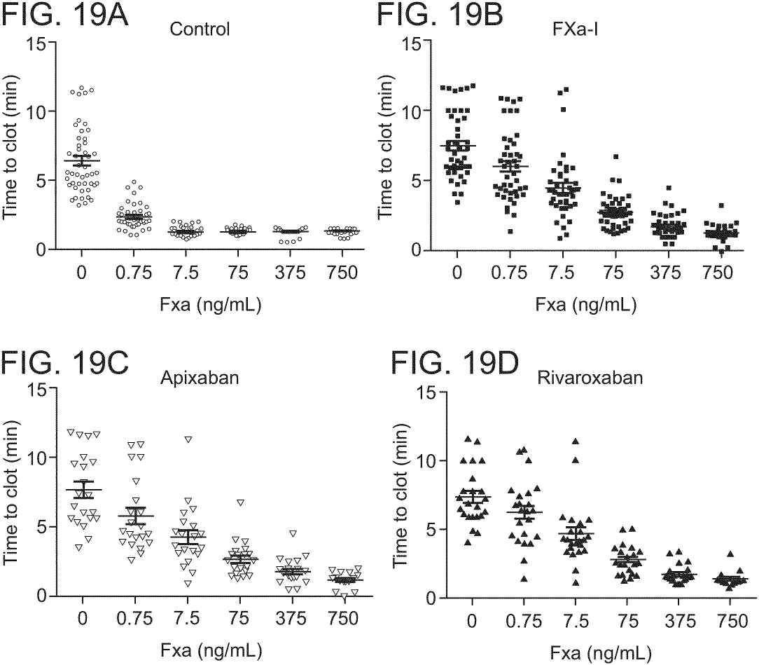

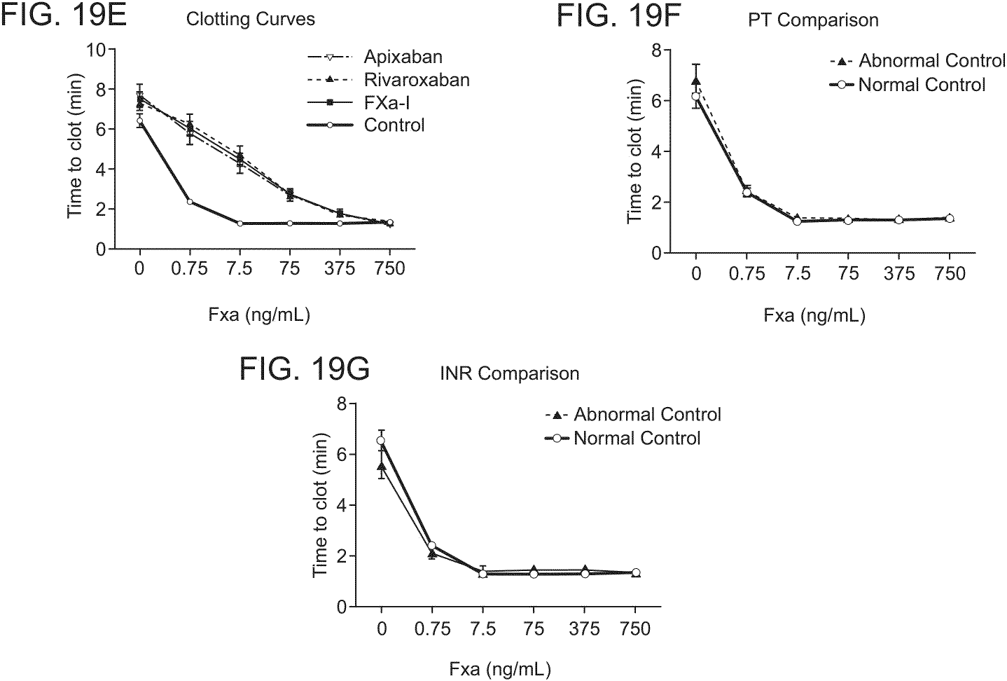

FIGS. 19A-19G illustrate example clotting time data and comparative clotting curves. Clotting times were compared at various agonist concentrations for all the patient groups to construct clotting curves. FIGS. 19A-19D show scatter plots demonstrating the mean and standard error bars of the clotting times at various agonist concentrations with respect to patients in different groups. FIG. 19E shows the mean clotting time with standard error bars of all patient groups, which are demonstrated on a single graph for comparison. All three FXa-I groups (Apixaban, Rivaroxaban, FXa-I) appear subjectively very different from the control group, with there being multiple concentrations where there are significant statistical differences between the controls and the total FXa-I, Rivaroxaban, and Apixaban groups. FIGS. 19F and 19G shows mean time to clot with standard error bars of the control group divided into patients with normal versus abnormal PT or INR, demonstrating no large difference in these tests between the different control groups.

### Example 20

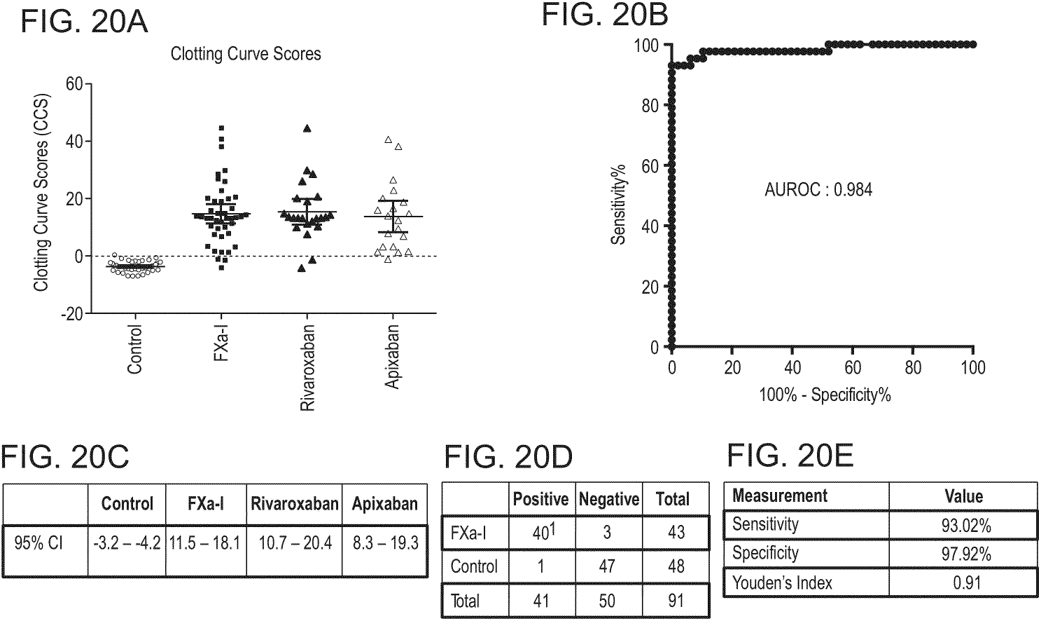

FIGS. 20A-20E illustrate Clotting Curve Score (CCS) analysis and evaluation of CCS utilization for the detection of FXa-I in patient samples. FIG. 20A shows a scatter plot with mean and standard error bars for CCS comparison between patient groups. Dotted line at CCS of 0 represents the chosen cut-off for the determination of whether there is FXa inhibition in the patient sample. FIG. 20B shows an ROC curve of utilizing the CCS scores to determine whether a patient has an FXa-I in their system. FIG. 20C provides descriptive statistics of the CCS for the different patient groups. FIGS. 20D and 20E illustrate evaluation of using the CCS for the determination of the accuracy of FXa-I detection.

### Example 21

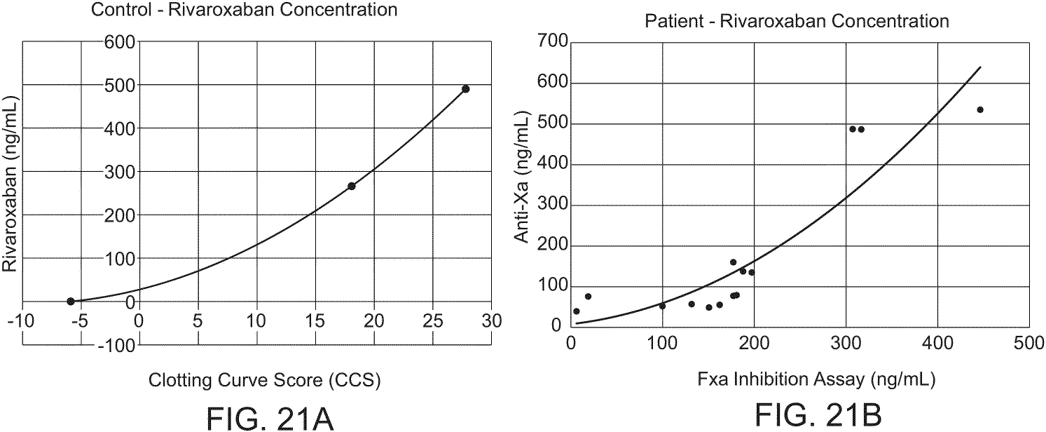

FIGS. 21A and 21B illustrate functional drug concentration calculation. Utilizing the CCS score calculated for each of the controlled spiked Rivaroxaban samples, a best-fit line was plotted for an equation that converted CCS into drug concentration, as shown in FIG. 21A. This equation was then applied to each of the CCS values for the patient samples evaluated in order to derive a functional concentration for each patient sample. These concentrations were directly compared to anti-Xa chromogenic assay-derived Rivaroxaban concentrations in each sample. Plotting these two values against each other demonstrated a good correlation between the anti-Xa concentration and the DOAC test concentration (R^2 = 0.827), as shown in FIG. 21B. Note that hemolyzed samples were not included in this direct comparison because it is known that hemolyzed, icteric, and lipemic plasma samples negatively affect the anti-Xa chromogenic assay concentrations.

In addition to identifying inhibition, as illustrated in the examples of FIGS. 21A and 21B, embodiments can be used to quantify the amount of inhibition.

### Example 22

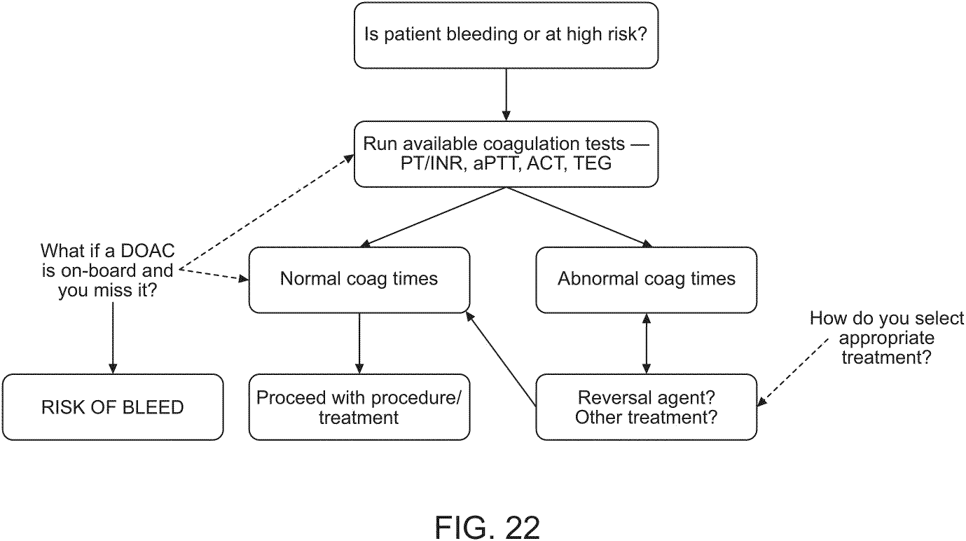

FIG. 22 illustrates a current decision-making paradigm if a patient is on a Direct Oral Anticoagulant (DOAC).

When a patient is at high-risk for a bleeding event or has an active bleed coagulation tests are ordered. These tests can include PT, INR, aPTT, ACT, TEG, or other currently available point-of-care tests. Abnormal clotting results on currently-available tests are non-specific for the presence of DOACs and leaves the healthcare worker guessing as to which treatment is the most appropriate for the patient. If the coagulation times are normal, due to the lack of sensitivity of these tests, the healthcare worker may miss the presence of a DOAC in the patient sample and proceed with treatment, putting the patient at an increased risk of bleeding.

### Example 23

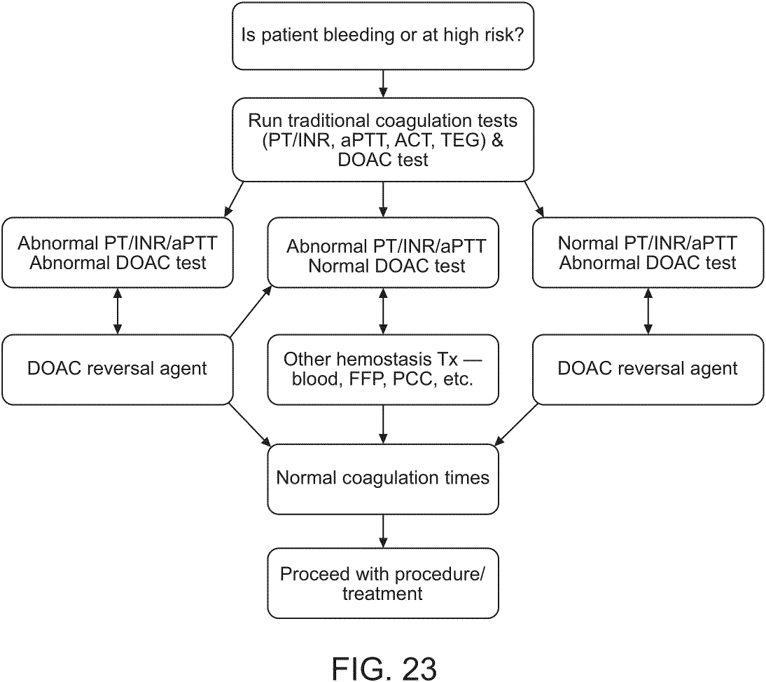

FIG. 23 illustrates an improved decision-making paradigm using embodiment(s) of the present invention if a patient is on a DOAC. Double arrows indicate possible iterative procedures. For example, if traditional coagulation tests show a patient has normal clotting times and the DOAC test according to an embodiment of the invention shows abnormal results, a DOAC reversal agent can be selected, based on test results, and administered to the patient. The patient can then re-tested, and, if still abnormal according to the DOAC, re-tested again, optionally after administrating a modified or different DOAC reversal agent. If the traditional coagulation tests are abnormal and the DOAC test is also abnormal, then the healthcare worker may choose the DOAC reversal agent or another treatment and re-test after administration of the reagent. If the traditional coagulation tests are abnormal and the DOAC test is negative for the presence of a DOAC then the healthcare worker has the information necessary to determine that another hemostatic treatment may be necessary.

### Example 24

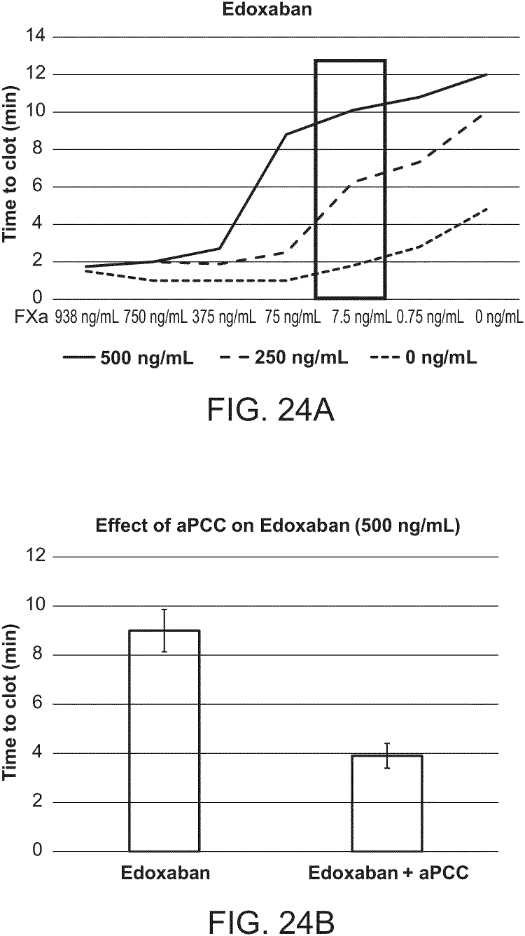

FIGS. 24A and 24B illustrate detection of the reversal of FXa inhibition following the addition of activated prothrombin complex concentrate (aPCC; FEIBA). FEIBA is a combination of activated factors administered to overcome FXa inhibitors in patients. Another example is Kcentra, which is inactive prothrombin complex concentrate. There are also specific FXa inhibitor reversal agents, such as coagulation factor Xa (recombinant), inactivated-zhzo. FIG. 24A demonstrates the expected clotting times for Edoxaban upon the addition of 7.5 ng/mL of FXa. FIG. 24B shows the change in clotting time with a plasma sample with 500 ng/mL of Edoxaban is treated with aPCC. This data demonstrates that the test according to an embodiment of the invention has utility to monitor the reversal or overcoming of the anticoagulant effect of these DOACs.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A microfluidic device for detecting coagulation, the microfluidic device comprising:
a first series of channels formed in a substrate, wherein the channels of the first series are coated with or contain a first coagulation factor at differing amounts across the channels of the first series such that the amount of the first coagulation factor differs by at least a factor of two between the channels of the first series; and
a second series of channels formed in the substrate, wherein the channels of the second series are coated with or contain a second coagulation factor at differing amounts across the channels of the second series such that the amount of the second coagulation factor differs by at least a factor of two between the channels of the second series;
wherein the channels of the first series and the channels of the second series have the same geometry, said geometry comprising an area where flow is disturbed to allow for clot formation, and
wherein the second coagulation factor acts upstream of the first coagulation factor in a coagulation pathway, and wherein
the first coagulation factor is in activated form,
the second coagulation factor is in activated form, or
both the first coagulation factor and the second coagulation factor are in activated form.

2. The microfluidic device according to claim 1, further comprising a control channel that is not coated with and does not contain any coagulation factor, wherein the control channel has the same geometry as the geometry of the channels of the first series and the channels of the second series.

3. The microfluidic device according to claim 1, wherein the first series of channels comprises at least three channels, and wherein the second series of channels comprises at least three channels.

4. The microfluidic device according to claim 1, wherein the first coagulation factor: is von Willebrand factor, prekallikrein (Fletcher factor), high-molecular-weight kininogen (HMWK) (Fitzgerald factor), fibronectin, antithrombin III, heparin cofactor II, protein C, protein
S, protein Z, Protein Z-related protease inhibitor (ZPI), plasminogen, alpha 2-antiplasmin, tissue
plasminogen activator (tPA), urokinase, plasminogen activator inhibitor-1 (PAI1), plasminogen
activator inhibitor-2 (PAI2), or Tissue Factor Pathway Inhibitor (TFPI).

5. The microfluidic device according to claim 1, wherein the first coagulation factor is selected from Factors I to XIII and activated forms thereof.

6. The microfluidic device according to claim 1, wherein the first coagulation factor is Factor IIa and the second coagulation factor is Factor Xa.

7. The microfluidic device according to claim 6, further comprising a control channel that is not coated with and does not contain any coagulation factor, wherein the control channel has the same geometry as the geometry of the channels of the first series and the channels of the second series.

8. The microfluidic device according to claim 1, wherein the first coagulation factor is Factor XIa and the second coagulation factor is Factor XIIa.

9. The microfluidic device according to claim 1, wherein the first coagulation factor is Factor Xa and the second coagulation factor is Factor XIa.

10. The microfluidic device according to claim 1, wherein the microfluidic device is configured to measure clot formation in each of the channels of the first series and in each of the channels of the second series at one or more fixed times.

11. The microfluidic device according to claim 1, wherein the microfluidic device is configured to measure clot formation in the channels of the first series and in the channels of the second series by one or more of: electrical impedance, change in flow velocity, change in pressure, thromboelastography, fluorescence detection using fluorescent fibrinogen, turbidity, infrared spectroscopy, image sensor, and light absorbance.

12. A microfluidic device for detecting coagulation, the microfluidic device comprising:
at least six channels formed in a substrate, each channel having the same geometry, said geometry comprising an area where flow is disturbed to allow for clot formation, wherein three of the at least six channels are each coated with or contain a different amount of a first coagulation factor such that the amount of the first coagulation factor differs by at least a factor of two between each of the three channels, and wherein another three of the at least six channels are each coated with or contain a different amount of a second coagulation factor such that the amount of the second coagulation factor differs by at least a factor of two between each of the other three channels, and
wherein the second coagulation factor acts upstream of the first coagulation factor in
a coagulation pathway, and wherein
the first coagulation factor is in activated form,
the second coagulation factor is in activated form, or
both the first coagulation factor and the second coagulation factor are in activated form.

13. The microfluidic device according to claim 12, further comprising a control channel that is not coated with and does not contain any coagulation factor.

14. The microfluidic device according to claim 12, wherein the first coagulation factor is Factor IIa and the second coagulation factor is Factor Xa.

15. The microfluidic device according to claim 14, further comprising a control channel that is not coated with and does not contain any coagulation factor.

16. The microfluidic device according to claim 12, wherein the first coagulation factor is Factor XIa and the second coagulation factor is Factor XIIa.

17. The microfluidic device according to claim 16, further comprising a control channel that is not coated with and does not contain any coagulation factor.

18. The microfluidic device according to claim 12, wherein the first coagulation factor is Factor Xa and the second coagulation factor is Factor XIa.

19. The microfluidic device according to claim 18, further comprising a control channel that is not coated with and does not contain any coagulation factor.

20. The microfluidic device according to claim 12, wherein the microfluidic device is configured to measure clot formation in each of the at least six channels at one or more fixed times.
